(19) United States
(12) Patent Application Publication
(10) Pub. No.: US 2023/0346931 A1
(43) Pub. Date: Nov. 2, 2023

(54) ALTERNATIVE SURFACTANTS AS STABILIZERS FOR THERAPEUTIC PROTEIN FORMULATIONS

(71) Applicant: Hoffmann-La Roche Inc., Little Falls, NJ (US)

(72) Inventors: Marc HEITZ, Basel (CH); Tarik KHAN, Basel (CH); Hannah Sophia KIENTZ, Offenburg (DE); Claudia MUELLER, Basel (CH); Janina Michelle PFAFF, Lörrach (DE)

(73) Assignee: Hoffmann-La Roche Inc., Little Falls, NJ (US)

(21) Appl. No.: 18/150,466

(22) Filed: Jan. 5, 2023

Related U.S. Application Data
(63) Continuation of application No. PCT/EP2021/068563, filed on Jul. 6, 2021.

(30) Foreign Application Priority Data
Jul. 7, 2020 (EP) 20184503.9

(51) Int. Cl. A61K 39/395; A61K 9/00; A61K 47/22; A61K 47/32; A61K 47/10; A61K 47/26; A61K 47/28

(52) U.S. Cl. CPC A61K 39/39591; A61K 9/0019; A61K 47/22; A61K 47/32; A61K 47/10; A61K 47/26; A61K 47/28

(57) ABSTRACT

The present invention relates to novel liquid pharmaceutical compositions comprising proteins, preferably antibodies as defined herein together with one or more surfactants selected from TPGS, PVA, T1107, Px338, Px407, TMN-6, 15-S-15, Chol-PEG, and SL.

### CROSS REFERENCE TO RELATED APPLICATIONS

This application is a continuation of International Application No. PCT/EP2021/068563, filed Jul. 6, 2021, which claims priority to European Patent Application No. 20184503.9, filed Jul. 7, 2020, which is incorporated herein by reference in its entirety.

### FIELD OF THE INVENTION

The present invention relates to the field of aqueous protein formulations, and in particular their stabilization against visible particle formation upon storage.

### BACKGROUND OF THE INVENTION

With Humulin® the first approved recombinantly produced biopharmaceutical drug in 1982 the success story of biologics as recombinant therapeutics, also called biopharmaceutics began [1]. Today biopharmaceutics are a major branch and a fast growing market with more and more products being in development [2]. Being amphiphilic macromolecules, proteins have the tendency to go into and accumulate at the interfaces, where they may aggregate. Therefore, proteins need to be protected from interfaces and interfacial stress to guarantee product stability. Interfacial stresses can arise during many stages of product manufacturing (e.g. freezing, thawing, filtration, pumping and filling operations) but also during shipment, storage, and administration to the patient [3-5]. Protein aggregation may lead to increased formation of sub-visible and/or visible particles or even potential loss of drug efficacy, or safety (i.e. in case of immunogenic reactions). Thus, preserving the formulation stability by preventing protein aggregation is crucial [6-8]. Different aggregation mechanisms and mitigation strategies to avoid protein aggregation were identified and described [9]. One general approach is the addition of excipients like surfactants to the formulation [10]. Surfactants are potent stabilizers against interfacial stresses leading to an inhibited or reduced adsorption of amphiphilic proteins at interfaces. Two mechanistic models on how surfactants protect proteins have been described: (1) the formation of surfactant-protein-complexes and (2) the primary mechanism of preferential competitive adsorption of surfactants at interfaces [4, 11-14].

Due to proven safety and efficacy, three nonionic surfactants have been used in the formulation development of marketed biologics: polysorbate 20, polysorbate 80 (also called Tweens®) or the triblock copolymer poloxamer 188 (Kolliphor® P, Pluronic® or Synperonic®). Polysorbates (PS) are known to be excellent stabilizers at both the air-water interface, present during agitation and stirring stresses [15, 16], and the silicone oil-water interface, predominantly found in pre-filled syringes, [17-19] but also during various other stresses like freeze/thawing and freeze-drying [20]. Therefore, this surfactant class is most widely used in commercial products [21, 22]. However, PS are also chemical inhomogeneous mixtures mainly consisting of an ethoxylated sorbitan backbone with up to three varying fatty acid side chains leading to substantial material variability between vendors and lots [23-25]. Apart from this, polysorbates can degrade by means of oxidation or hydrolysis which may lead to problems due to: (1) reduced protection or proteins against interfacial stresses which might be accompanied by protein particle formation and/or (2) negative impact of PS degradants on the stability of the protein [25-27]. Further studies on PS degradation reported an enzymatic cleavage of the ester bond, presumably caused by impurities from host cell proteins [28, 29]. Both the hydrolytic and the enzymatic pathway can develop free fatty acid (FFA) degradants with limited solubility and the tendency to form sub-visible and visible particles [27, 30, 31].

In contrast, poloxamers 188 (Px) reported to be more stable, consists of two hydrophilic polyethylene oxide (PEO) units joined by a more hydrophobic polypropylene oxide (PPO) middle block [32, 33]. The more hydrophilic character (HLB>24) of Px188 was hypothesized to contribute to an increased Fc-fusion protein adsorption the silicone oil water interface in prefilled syringes compared to PS80 (HLB=15.0) [19]. Px188 has also been reported to present a greater risk of protein-silicone oil particle formation in vials. This may present a challenge for the use of Px188 as stabilizing agent particularly in prefilled syringes (PFS), depending on protein molecular properties, amount of silicone oil, and other features of the drug product configuration, since these devices use silicone oil as lubricant which may account for most of the particles detected in biopharmaceutical products stored in PFS [8, 34, 35].

Recently some new molecules have been suggested as alternative surfactants. Maggio reported that alkyl saccharides were able to stabilize Interferons, and monoclonal antibodies (mAbs) comparable to PS [36, 37]. Furthermore, alkyl saccharides were reported to be stable against oxidative degradation [38]. However, compared to other surfactants the hemolytic activity of alkyl saccharide surfactants is higher—in particular n-dodecyl-β-d-maltopyranoside (DDM)—which makes their therapeutic use more challenging [39]. Less pronounced hemolytic activity was reported from Schiefelbein et al. for their synthesized trehalose-based surfactants which also showed promising stabilizing effects for human growth hormone (hGH) upon shaking [40]. Katz et al. synthesized a novel amino acid based surfactant called FM100 and tested its ability to protect IgG and abatacept against agitation stresses in comparison with polysorbate 20 and 80 as well as poloxamer 188. They found FM100 to stabilize an interface faster than all three other surfactants with an increased stabilization of model proteins against agitation-induced aggregation [41, 42]. Nevertheless, implementation in clinical or commercial formulations of biologics for any of the above named surfactants has not yet been reported. Other classes of nonionic surfactants often described as interfacial stabilizers are primary alcohol ethoxylates like Brij® or alkylphenol ethoxylates like Triton™ X [43, 44]. However, most of the before mentioned molecules are either not approved for parenteral use or represent safety concerns for repeated and common parenteral administration.

Therefore, there is a need for alternative surfactants which do not show liabilities regarding intrinsic stability and adsorption behavior to pharmaceutically relevant interfaces. In particular, there remains a need to investigate on novel/ alternative surfactants to mitigate existing liabilities of established surfactants for parenteral administration in order to expand the toolbox for formulation development while guaranteeing optimal drug product stability.

The present invention solves this problem by suggesting known surfactants for a novel use as stabilizers in therapeutic protein formulations. More particularly, the present inventors carried out a comprehensive evaluation of structural compositions of surfactants needed to possess good protein stabilizing effects at relevant interfaces, and to be less prone for enzymatic degradation. The present inventors investigated on surfactants with a broad structural variety regarding hydrophobic as well as hydrophilic molecular parts (see FIG. 1). The present inventors also implemented screening tools to analyze the surfactants in terms of stability against enzymatic degradation and their impact on the thermal stability of a model mAb. To exclude negative impact on long-term stability, samples were stored for up to 18 months at 5° C., 25° C., as well as 3 months at 40° C. and analyzed with regard to changes in visible and sub-visible particles, turbidity, color, pH, mAb monomer and mAb charge. Controls using PS20 and Px188 were run in parallel.

### BRIEF DESCRIPTION OF THE FIGURES

FIG. 1: Graphical representation of all alternative surfactants tested within this work. The structures are clustered according to their lipophilic part in 4 subgroups: i) acyl-, ii) alkyl-, iii) sterol-group and iv) others. Additionally, the molecules were differentiated by their hydrophilic head group as: i) polyethylene oxide (PEO) based and ii) sugar based surfactants. Marked excipients (*) have been used within parenteral marked product within the FDA and EMA [45, 46].

FIG. 2: Representative depiction of a PS20 RP-HPLC chromatogram classified into hydrophilic non-esterified (1) and lipophilic esterified (2) fractions before (-) and after enzymatic digestion (- --). The main peak of the esterified fraction (lipophilic) and the free, non-esterified (hydrophilic) peak were integrated and evaluated.

FIG. 3: Degree of surfactant degradation. Normalized ester main peak area is shown before ( [symbol] ) and after incubation with 0.25 ( [symbol] ) and 0.5 ( [symbol] ) mg/mL PCL/CALB lipase mixture (1:1), or 0.1 mmol sodium hydroxide ( [symbol] ) respectively. ** no ester main peak was observed, complete degradation.

FIG. 4: Thermal conformational stability of mAb in presence of surfactants. Figures show the average value of three individual measurements of Tₒₙ, (A) and Tₘ₁ (B). Surfactants with colored values demonstrate a considerable decrease of thermal stability properties compared to the control formulation without surfactant (-).

FIG. 5: Cumulative count of sub-visible particles ≥10 μm per mL for control formulation ( [symbol] ) without surfactant and formulations with ( [symbol] ) 0.1 and ( [symbol] ) 1 mg/mL surfactant. SVP count after horizontal shaking at 5° C. (A) and 25° C. (B), 5 constitutive freeze-thaw cycles (C) and after storage at 40° C. for 12 weeks (D) compared to initial values (♦). Formulations with SVP in the upper segment of the discontinuous y-axis exceeded USP <787> criteria of maximum 6,000 particles ≥10 μm per container.

FIG. 6: Soluble aggregate levels, given as increase in HMWS (area %) after different stress conditions and surfactant concentrations ( [symbol] 0, [symbol] 0.1 and [symbol] 1 mg/mL): horizontal shake stress for 7 days at 200 rpm at (A) 5° C. or (B) 25° C., (C) 5 constitutive freeze-thaw cycles, and (D) storage at 40° C. for 12 weeks.

FIG. 7: Cumulative count of sub-visible particles ≥2 μm per mL of mAb formulations ( [symbol] ) and placebo ( [symbol] ) at initial time point. Samples contained either (A) 0.1 mg/mL or (B) 1 mg/mL surfactant.

FIG. 8: Representative monomer loss by means of SE-HPLC for formulations stored at 25° C. containing (A) 0.1 mg/mL or (B) 1 mg/mL surfactant. In this graph only formulations with a considerable change in main peak area are shown ( [symbol] SL and [symbol] CS20) and compared to standard surfactants PS20 ( [symbol] ) and Px188 ( [symbol] ).

FIG. 9: Representative decrease of the mAb main peak area measured by IE-HPLC. Formulations were stored at 5° C. (open symbols), 25° C. (half filled symbols) and 40° C. (filled symbols) containing (A) 0.1 mg/mL or (B) 1 mg/mL surfactant. Here, only CS20 (triangle) formulations which showed a considerable decrease of the main peak area are presented and compared to standard surfactants PS20 (square) and Px188 (circle).

FIG. 10: Physicochemical and structural characteristics of evaluated surfactants and their current field of application.

FIG. 11: Formulation attributes for formulations containing no surfactant and acyl-based surfactants for mAb 1.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1.
^{d}Values are given as average of three individual measurements (standard deviation <0.4).
^{e}Shaking experiments show average value of 2-3 analyzed vials. Surface tension of 20 mM His-HCl buffer is 73 mN·m−1. Class ii, iii, iv results were marked in grey. The darker the color the higher the class and poorer the outcome of the stress test.
n.a.=not analyzed

FIG. 12: Formulation attributes for formulations containing alkyl-based surfactants for mAb 1.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1.
^{d}Values are given as average of three individual measurements (standard deviation <0.4).
^{e}Shaking experiments show average value of 2-3 analyzed vials. Surface tension of 20 mM His-HCl buffer is 73 mN·m−1. Class ii, iii, iv results were marked in grey. The darker the color the higher the class and poorer the outcome of the stress test.
n.a.=not analyzed.

FIG. 13: Formulation attributes for formulations containing sterol-based surfactants for mAb 1.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1.
^{d}Values are given as average of three individual measurements (standard deviation <0.4).
^{e}Shaking experiments show average value of 2-3 analyzed vials. Surface tension of 20 mM His-HCl buffer is 73 mN·m−1. Class ii, iii, iv results were marked in grey. The darker the color the higher the class and poorer the outcome of the stress test.
n.a.=not analyzed.

FIG. 14: Formulation attributes for formulations containing surfactants of class “others” for mAb 1.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
Color is categorized in 4 groups according to the color scale values of Ph.Eur. 2.2.2: i) 9-7, ii) 6-5, iii) 4-3 and iv) 2-1.
^{d}Values are given as average of three individual measurements (standard deviation <0.4).
^{e}Shaking experiments show average value of 2-3 analyzed vials. Surface tension of 20 mM His-HCl buffer is 73 mN·m−1. Class ii, iii, iv results were marked in grey. The darker the color the higher the class and poorer the outcome of the stress test.
n.a.=not analyzed.

FIG. 15: Formulation attributes for formulations containing no surfactant and acyl-based surfactants for mAb 2 and 3.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1. Class ii, iii, iv results were marked in grey. The darker the color the higher the class and poorer the outcome of the stress test.

FIG. 16: Formulation attributes for formulations containing alkyl-based surfactants for mAb 2 and 3.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1. Class ii, iii, iv results were marked in grey. The darker the color the higher the class and poorer the outcome of the stress test.

FIG. 17: Formulation attributes for formulations containing sterol-based surfactants for mAb 2 and 3.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1. Class ii, iii, iv results were marked in grey. The darker the color the higher the class and poorer the outcome of the stress test.

FIG. 18: Formulation attributes for formulations containing surfactants of class “others” for mAb 2 and 3.
^{a}Visible particles are categorized in the 4 groups i) 0 particles, ii) 1-5 particles, iii) 6-10 particles and iv) >10 particles.
^{b}Turbidity is categorized in the 4 groups i) 0-3 NTU, ii) >3-6 NTU, iii) >6-18 NTU and iv) >18 NTU.
^{c}Color is categorized in 4 groups according to the color scale values of Ph.Eur. 2.2.2: i) 9-7, ii) 6-5, iii) 4-3 and iv) 2-1. Class ii, iii, iv results were marked in grey. The darker the color the higher the class and poorer the outcome of the stress test.

### DETAILED DESCRIPTION OF THE INVENTION

Surfactants approved for parenteral use possess two structural weaknesses: (1) ester linkers within the molecule makes them prone for enzymatic degradation by host cell proteins (HCPs) which may result in the formation of visible free fatty acid particles or (2) a charge which is reported to lead to destabilizing of the mAb presumably by charged-charged interactions [56]. Therefore, two screening tools were investigated to test on these structural components. This allowed the quick identification of potential alternative surfactants and facilitated the evaluation of a large number of possible candidates.

With the fast growing biologics market the development of screening tools to forecast the long-term stability of proteins, especially antibodies gained in importance. Several biophysical characterization techniques predicting protein stability are known [47, 48]. Amongst others, the maximization of the conformational stability is regarded to have a high impact maintaining long-term drug product quality by preventing unfolding and aggregation of therapeutic proteins.

Applied screening techniques are DSC (Differential Scanning Calorimetry) or measuring the intrinsic protein fluorescence under isothermal chemical denaturation (ICD) or thermal denaturation conditions by means of nanoDSF (Differential Scanning Fluorimetry) [49]. Since pH, buffer system and excipient composition can influence proteins' intrinsic conformational stability these screenings are often performed in early stage formulation development [50].

As second pre-screening tool, nano-DSF measurements was investigated. For the therapeutic relevant surfactant concentration of ≤1 mg/mL, most tested alternate surfactants only showed slight changes in Tₒₙ and Tₘ.

The present inventors established an easy and quick method to evaluate the ester stability of various surfactants against enzymatic digestion. It was found by the present inventors that surfactants vary in the degree of enzymatic degradation depending on the size of their lipophilic backbone, presumably by steric interference with the enzymatic active center.

Moreover, the present inventors established a structure activity relationship for the sterol-based surfactants upon interfacial stresses but also during long-term storage. Revealing small and flexible structures are more effective in protein stabilization upon fast changes at the interface e.g. during shaking compared to bulky surfactants. A similar group behavior was also found for the polymeric surfactants: poloxamer, tetronic, and polyvinylalcohol upon agitation stresses.

The present invention surprisingly identified the surfactants TPGS, PVA, T1107, Px338, Px407, TMN-6, 15-S-15, Chol-PEG, and SL as showing comparable or superior protein stabilizing effects than the established PS20, PS80 and Px188 in liquid composition comprising said proteins.

Therefore, in one embodiment, the present invention provides a liquid pharmaceutical composition comprising a protein and one or more surfactant(s) selected from TPGS, PVA, T1107, Px338, Px407, TMN-6, 15-S-15, Chol-PEG, and SL.

In another embodiment, the present invention provides an aqueous pharmaceutical composition comprising a protein and one or more surfactant(s) selected from TPGS, PVA, T1107, Px338, Px407, TMN-6, 15-S-15, Chol-PEG, and SL.

In another embodiment, the present invention provides an aqueous pharmaceutical composition comprising a protein and one or more surfactant(s) selected from TPGS, PVA, T1107, Px338, Px407, TMN-6, 15-S-15 and SL.

In another embodiment, the present invention provides an aqueous pharmaceutical composition comprising a protein and one or more surfactant(s) selected from TPGS, PVA, T1107, Px338, Px407 and SL.

In another embodiment, the present invention provides an aqueous pharmaceutical composition comprising a protein and one or more surfactant(s) selected from TMN-6 and 15-S-15.

In another embodiment, the present invention provides any of the aforementioned compositions, wherein the protein is a pharmaceutically active ingredient. In one aspect said composition is for use in treating a disease in a patient in need of treatment.

In another embodiment, the present invention provides any of the aforementioned compositions, wherein the protein is an antibody; or an immunoconjugate; or an antibody fragment.

In another embodiment, the present invention provides any of the aforementioned compositions, wherein the protein is an antibody comprised in any of the antibody products as defined herein.

In another embodiment, the present invention provides any of the aforementioned compositions, further comprising pharmaceutically acceptable excipients or carriers.

In another embodiment, the present invention provides any of the aforementioned compositions, wherein the surfactant(s) is/are present in a concentration of ≤1 mg/mL; or in a concentration range from 0.001 mg/mL to 0.01 mg/mL; or from 0.01 mg/mL to 0.1 mg/mL; or from 0.1 mg/mL to 1.0 mg/mL. In one embodiment the surfactants in accordance with the present invention are TPGS and/or PVA at a concentration of 1 mg/mL. In another embodiment, the surfactants in accordance with the present invention are 15-S-15 and/or TMN-6 at a concentration of 1 mg/mL or 0.1 mg/mL

In another embodiment, the present invention provides any of the aforementioned compositions, wherein the proteins are present in any concentration known to the person of skill in the art to be applicable for aqueous protein, or antibody formulations. In one embodiment, especially where the protein is an antibody approved for use as a human medicament, the antibodies are present at any of their approved concentrations. Information about said approved concentrations is easily available to the skilled person, for example, on the package insert or the summary of product characteristics (SmPC) for a given drug. In another embodiment, a protein, especially an antibody, is present in a composition in accordance with the present invention in a concentration from 5-200 mg/ml, or 5-100 mg/ml, or 10-25 mg/ml.

In yet another embodiment, the present invention provides the use of one or more surfactants selected from TPGS, PVA, T1107, Px338, Px407, TMN-6, 15-S-15, Chol-PEG, and SL in the manufacture of a liquid pharmaceutical composition further comprising a protein.

In yet another embodiment, the present invention provides the use of one or more surfactants selected from TPGS, PVA, T1107, Px338, Px407, TMN-6, 15-S-15 and SL in the manufacture of a liquid pharmaceutical composition further comprising a protein.

In yet another embodiment, the present invention provides the use of one or more surfactants selected from TPGS, PVA, T1107, Px338, Px407 and SL in the manufacture of a liquid pharmaceutical composition further comprising a protein.

In yet another embodiment, the present invention provides the use of one or more surfactants selected from TMN-6 and 15-S-15 in the manufacture of a liquid pharmaceutical composition further comprising a protein.

In another aspect, the present invention provides any of the aforementioned uses for the manufacture of an aqueous pharmaceutical composition comprising an antibody as defined herein. In one aspect said composition is an approved medicament, comprising an antibody or a multi- or bispecific antibody as active ingredient. In another embodiment, the present invention provides the use of one or more surfactants selected from TPGS, PVA, T1107, Px338, Px407, TMN-6, 15-S-15, Chol-PEG, and SL for stabilizing a protein and preventing the formation of visible particles in a liquid pharmaceutical composition comprising said protein, upon storage. In another embodiment, the liquid pharmaceutical composition comprises one or more proteins as active ingredient.

Still, in another embodiment, the present invention provides one or more surfactants selected from TPGS, PVA, T1107, Px338, Px407, TMN-6, 15-S-15, Chol-PEG, and SL for use in any of the liquid pharmaceutical compositions as disclosed herein before. In one embodiment, said use means to stabilize the protein comprised in said liquid pharmaceutical composition and to prevent the formation of visible particles in said composition upon storage.

In yet another embodiment, the present invention provides one ore more surfactants as defined herein, preferably TPGS, PVA, T1107, Px338, Px407, TMN-6, 15-S-15 and/or SL to replace PS20, PS80 or Poloxamer 188 in commercial antibody preparations. In another embodiment, 15-S-15 can be used to replace any of PS20, PS80 or Poloxamer 188 in an aqueous pharmaceutical composition comprising an antibody as defined herein. In another embodiment TPGS, Px338, Px407, PVA, T1107, TMN-6 and SL can be used to replace Poloxamer 188 in an aqueous pharmaceutical composition comprising an antibody as defined herein.

In yet another embodiment, the present invention provides the use of one or several surfactants as defined herein for the manufacture of a medicament. In one aspect, said medicament is an aqueous pharmaceutical preparation comprising any active ingredient which requires stabilization by surfactants for its authorized application. In another aspect the one or several surfactant(s) is/are independently selected from TPGS, PVA, T1107, Px338, Px407, TMN-6, 15-S-15 and SL.

In yet another aspect, the present invention provides the screening methods, alone or in combination, disclosed herein for identifying the surfactants in accordance with the present invention. In one embodiment, the screening methods are as disclosed in the accompanying working examples.

The antibody designated “mAb 1” herein is the antibody with the INN pertuzumab, Pertuzumab is commercially available, for example under the tradename PERJETA®. Pertuzumab is, for example, also disclosed in EP 2 238 172 B1. Therefore, in one embodiment, “pertuzumab” (or “rhuMAb 2C4”) refer to an antibody comprising the variable light and variable heavy amino acid sequences in SEQ ID Nos. 3 and 4, respectfully as disclosed in EP 2 238 172 B1. Where Pertuzumab is an intact antibody, it comprises the light chain and heavy chain amino acid sequences in SEQ ID Nos. 15 and 16, respectively as disclosed in EP 2 238 172 B1.

The antibody designated “mAb 2” herein is the antibody with the INN obinutuzumab. Obinutuzumab is commercially available, for example under the tradename GAZYVA®/GAZYVARO®. Sequence information for obinutuzumab is published, for example by the WHO on its list of recommended INN's (List 65, WHO Drug Information, Vol. 25, No 1, 2011). Additional information for obinutuzumab is, for example, also available in WO2005/044859 (B-HH6 is the heavy chain construct, and B-KV1 the light chain construct). See also Tables 2 and 3 in WO2005/044859 for sequence information.

The antibody designated “mAb 3” herein is an investigational bi-specific antibody-fragment which is in clinical trials.

The term “surfactant” as used herein means TPGS, PVA, T1107, Px338, Px407, TMN-6, 15-S-15, Chol-PEG, and SL.

TPGS is Tocofersolan (D-α-Tocopherol polyethylene glycol succinate)

PVA is Poly(vinyl) alcohol 4-88

T1107 is Tetronic® 1107 (Ethylenediamine tetrakis(propoxylate-block-ethoxylate) tetrol)

Px338 is Kolliphor® P 338 (Poly(ethylene glycol)-block-poly(propylene glycol)-block-poly(ethylene glycol)

Px407 is Kolliphor® P 407 (Poly(ethylene glycol)-block-poly(propylene glycol)-block-poly(ethylene glycol)

TMN-6 is Tergitol™ TMN-6 (Branched secondary alcohol ethoxylate with 8 EO units)

15-S-15 is Tergitol™ 15-S-15 (Secondary alcohol ethoxylate with 15 EO units)

Chol-PEG is mCholesterol-PEG2000 and

SL is REWOFERM SL ONE (Aqueous solution of sophorolipids (17[2-O-(6-O-Acetyl-beta-D-glucopyranosyl)-6-O-acetyl-beta-D-glucopyranosyloxyl]-9-octadecenoic acid) lactone- and acid form).

The term “storage” as used herein means keeping a liquid pharmaceutical preparation under conditions usually applied by the person of skill in the art. In one aspect said storage involves a time of up to 6 months, or 12 months, or 18 months, or 24 months, or 30 months. In another aspect said storage involves keeping said liquid pharmaceutical composition up to its shelf life as approved by regulatory authorities under conditions (such as e.g. temperature) as also approved by such regulatory authority. In one aspect such shelf life and storage conditions can, for example, be found in the package insert accompanying an approved protein based drug.

The term “liquid pharmaceutical composition” preferably means an aqueous composition, formulation or dosage form for pharmaceutical use. In one embodiment said liquid pharmaceutical compositions are for parenteral application of therapeutic proteins. In another embodiment, the liquid pharmaceutical compositions in accordance with the present invention comprise one or more therapeutic proteins together with pharmaceutically acceptable excipients or carriers. Such excipients are generally known to a person of skill in the art. In one embodiment, the term “excipient” refers to an ingredient in a pharmaceutical composition or formulation, other than an active ingredient, which is nontoxic to a subject. An excipient includes, but is not limited to, a buffer, stabilizer including antioxidant, or preservative.

The term “pharmaceutical composition” refers to a preparation, formulation or dosage form which is in such form as to permit the biological activity of an active ingredient contained therein to be effective, and which contains no additional components which are unacceptably toxic to a subject to which the pharmaceutical composition would be administered.

The term “pharmaceutically acceptable carrier” refers to an ingredient in a pharmaceutical composition or formulation, other than an active ingredient, which is nontoxic to a subject. A pharmaceutically acceptable carrier includes, but is not limited to an excipient as defined herein.

The term “buffer” is well known to a person of skill in the art of organic chemistry or pharmaceutical sciences such as, for example, pharmaceutical preparation development. Buffer as used herein means acetate, succinate, citrate, arginine, histidine, phosphate, Tris, glycine, aspartate, and glutamate buffer systems. Furthermore, within this embodiment, the histidine concentration of said buffer is from 5 to 50 mM.

The term “stabilizer” is well known to a person of skill in the art of organic chemistry or pharmaceutical sciences such as, for example, pharmaceutical preparation development. A stabilizer in accordance with the present invention is selected from the group consisting of sugars, sugar alcohols, sugar derivatives, or amino acids. In one aspect the stabilizer is (1) sucrose, trehalose, cyclodextrines, sorbitol, mannitol, glycine, or/and (2) methionine, and/or (3) arginine, or lysine. In still another aspect, the concentration of said stabilizer is (1) up to 500 mM or (2) 5-25 mM, or/and (3) up to 350 mM, respectively

The term “protein” as used herein means any therapeutically relevant polypeptide. In one embodiment, the term protein means an antibody. In another embodiment, the term protein means an immunocunjugate.

The term “antibody” herein is used in the broadest sense and encompasses various antibody classes or structures, including but not limited to monoclonal antibodies, polyclonal antibodies, multispecific antibodies (e.g., bispecific antibodies), and antibody fragments so long as they exhibit the desired antigen-binding activity. In one embodiment, any of these antibodies is human or humanized. In another embodiment, the antibody in accordance with the present invention is a human or humanized, mono- or bispecific antibody, preferably a monoclonal antibody of an IgG class. Said antibody may also comprise a combination of structural elements from different IgG classes or be conjugated to a moiety with pharmacological activity such as, for example, a cytotxic agent or a receptor ligand. In another aspect, the antibody is an “antibody product” selected from alemtuzumab (LEMTRADA®), atezolizumab (TECENTRIQ®), bevacizumab (AVASTIN®), cetuximab (ERBITUX®), panitumumab (VECTIBIX®), pertuzumab (PERJETA®, 2C4, Omnitarg), trastuzumab (HERCEPTIN®), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, daclizumab (ZENAPAX®), dalotuzumab, denosumab (PROLIA®, XGEVA®), eculizumab (SOLIRIS®), efalizumab, epratuzumab, erlizumab, emicizumab (HEMLIBRA®), felvizumab, fontolizumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralivizumab, ranibizumab (LUCENTIS®), reslivizumab, reslizumab, resyvizumab, robatumumab, rontalizumab, rovelizumab, ruplizumab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizumab, urtoxazumab, ustekinumab (STELARA®), vedolizumab (ENTYVIO®), visilizumab, zanolimumab, zalutumumab, obinutuzumab (GAZYVA®). In yet another embodiment, the antibody is pertuzumab or obinutuzumab.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called a, d, e, g, and m, respectively. The light chain of an antibody may be assigned to one of two types, called kappa (κ) and lambda (λ), based on the amino acid sequence of its constant domain.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived front a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

In one aspect, the term “long-term”, also in connection with “storage” or “stability” as used herein, means until the end of the authorized shelf life for any commercial antibody product as defined herein. In another aspect the term “long-term” means up to 5 years, or up to 3 years, or up to 24 months, or up to 18 months, or up to 12 months, or up to 6 months, or up to 3 months for the antibodies as defined herein in general. The term “storage” involves conditions such as, for example, temperature and humidity, which are usually required to store an antibody, especially any of the authorized antibody products as defined herein. Such conditions are well known to the skilled person. Reference to such conditions can, for example, be found on the package inserts or the summaries of product characteristics (SmPC's) of the commercial products among the antibody products as defined herein.

### A. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al.,

Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies front transgenic animals, see Lonberg, Nat.

Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### C. Antibody Derivatives

In certain aspects, an antibody provided herein may he further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### D. Immunoconjugates

The invention also provides immunoconjugates comprising an antibody herein conjugated (chemically bound) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science :238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are comercially available (e.g., from Pierce Biotechnology, Inc., Rockford, IL., U.S.A).

### E. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell. et al., J. Mol. Biol. 270:26 (1997)). Multi-specific antibodies may also be made by engineering electrostatic steering effects for making antibody Fc-heterodimeric molecules (see, e.g., WO :2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to two different antigens, or two different epitopes of the same antigen (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab frament comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also he engineered by introducing charged or non-charged amino acid mutations into domain interfaces to direct correct. Fah pairing. See e.g., WO 2016/172485.

Various further molecular formats for multispecific antibodies are known in the art and are included herein (see e.g., Spiess et al., Mol Immunol 67 (2015) 95-106).

### F. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with. heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can he comprised in one or more nucleic acid molecules or expression vectors.

Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B.K.C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived front multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B.K.C. (ed.), Humana Press, Totowa, NJ (2004), pp. 255-268.

The invention will now be further illustrated by the following, non-limiting working examples.

### EXAMPLES

### Material and Methods

### Materials

PCL (Lipase from Pseudomonas cepacia) and CALB (Lipase B Candida antarctica, recombinant from Aspergillus oryzae) were purchased from Sigma Aldrich (Steinheim, Germany). The model IgG₁ monoclonal antibodies used was provided by F. Hoffmann-La Roche (Basel, Switzerland) and respectively formulated at 25 mg/mL in 20 mM His-HCl buffer (Ajinomoto, Tokyo, Japan) with 240 mM sucrose (Pfanstiehl Inc., Illinois, USA) at pH 7.0 (mAb 1), 25 mg/mL in 17 mM His-HCl buffer with 240 mM sucrose at pH 6.0 (mAb 2) and 10 mg/mL in 10 mM His-HCl buffer with 240 mM sucrose at pH 6.0 (mAb 3).

The screened surfactants were provided by: Kolliphor® HS 15 (HS15, BASF, Ludwigshafen, Germany), Kolliphor® RH 40 (RH40, BASF, Ludwigshafen, Germany), polysorbate 20 (PS20; Croda International, Snaith, UK), polysorbate 80 HX2 (PS80; NOF Corporation, Tokyo, JP), 1,2-distearoyl-sn-glycero-3-phosphoethanolamine-N-[methoxy(polyethylene glycol)-2000](ammonium salt) (mPEG-DSPE; Avanti Polar Lipids, Alabaster, Alabama), Rewoferm® SL ONE (SL; Evonik Industries, Essen, Germany), Kolliphor® CS20 (CS20; BASF, Ludwigshafen, Germany), Tergitol™ 15-S-15 (15-S-15; Sigma Aldrich, Steinheim, Germany), Tergitol™ TMN-6 (TMN-6; Sigma Aldrich, Steinheim, Germany), Ecosurf™ EH-9 (EH-9; Sigma Aldrich, Steinheim, Germany), nonanoyl-N-methylglucamide (MEGA-9; Sigma Aldrich, Steinheim, Germany) and decanoyl-N-hydroxyethylglucamide (HEGA-10; Anatrace, Maumee, Ohio), sodium deoxy cholate (NaDC; Sigma Aldrich, Steinheim, Germany), sodium glycocholate hydrate (NaGC; Sigma Aldrich, Steinheim, Germany), Chobimalt/Cholestrol-β-D-Maltopryanosyl-(1 [symbol] 6)-β-D-Maltopyranoside (Chobi; Anatrace, Maumee, Ohio), mCholesterol-PEG₂₀₀₀ (Chol-PEG; Nanocs Inc., New York), Deoxy-BigCHAP (DBC; Toronto Research Chemicals, North York, Canada), α-tocopheryl-polyethylene-glycol-1000-succinate (TPGS, Toronto Research Chemicals, North York, Canada); Kolliphor® P 188 (Px188; BASF, Ludwigshafen, Germany), Kolliphor® P 338 (Px338; BASF, Ludwigshafen, Germany), Kolliphor® P 407 (Px407; BASF, Ludwigshafen, Germany), Tetronic® 1107 (T1107; BASF, Ludwigshafen, Germany), or polyvinyl alcohol 4-88 (PVA; Merck KGaA, Darmstadt, Germany).

All other reagents like methanol (MeOH), sodium hydroxide (NaOH) and ammonium acetate were of analytical grade and obtained from Merck KGa, Darmstadt, Germany

### Methods

### Measurement of Surfactant Stability Against Enzymatic Ester Hydrolysis

0.4 mg/mL of each surfactant were incubated in 20 mM ammonium acetate buffer pH 5.5 for 6 h at room temperature with a total of either 0.25 or 0.5 mg/mL of a 1:1 lipase mixture of PCL and CALB. Both belong to the group of carboxylester hydrolases, previously reported as important impurities from bioprocessing able to hydrolyze PS20 [51]. In case of no or negligible enzymatic degradation a chemical ester hydrolysis using NaOH was performed as positive control. For this, the surfactants were incubated for 6 h at room temperature with 0.1 mmol NaOH.

Surfactants degradation was followed by reverse phase high performance liquid chromatography (RP-HPLC) using a Waters Alliance 2695 instrument equipped with a Waters 2424 Evaporative Light Scattering Detector (ELSD) (Waters, Milford, USA) which used nitrogen as carrier gas with a pressure of 25 psi, and a drift tube temperature of 95° C. A Phenomenex Luna® C18(2) 100 Å (150×4.6 mm, 5 μm) column (Phenomenex, Torrence, USA) was used as stationary phase. Analysis of the surfactants was performed using a binary gradient elution with the program presented in Table 1 at a constant flow rate of 0.7 ml/min, a 25 μl injection volume, a sample temperature of 5° C., and a column temperature of 35° C. Eluent A consisted of 2% aqueous solution of acetic acid and eluent B of 2% acetic acid in MeOH.

TABLE 1
Gradient program for RP-HPLC surfactant analysis.

| Time (min) | Flow rate (mL/min) | % Eluent A | % Eluent B |
|---|---|---|---|
| 2.00 | 0.70 | 95 | 5 |
| 2.01 | 0.70 | 40 | 60 |
| 8.50 | 0.70 | 40 | 60 |
| 8.51 | 0.70 | 0 | 100 |
| 20.50 | 0.70 | 0 | 100 |
| 20.51 | 0.70 | 95 | 5 |
| 22.00 | 0.70 | 95 | 5 |

A typical chromatogram is shown in FIG. 2. As reported in literature the peaks were clustered into hydrophilic/non-esterified fraction (1) as well as into lipophilic, esterified fraction (2) [52-54]. Data processing was performed using Empower® 3 software. For a better comparison of the different surfactants, the degree of surfactant degradation was reported as normalized ester main peak area, for which the initial ester main peak area prior to degradation was set to 100%.

Results are given as the average of three individual measurements with a standard deviation of ≤0.15.

### Evaluation of Thermal Conformational Protein Stability in Presence of Surfactants

Conformational protein stability was investigated with the Prometheus NT.Plex (NanoTemper Technologies GmbH, München, Germany). The device allows the label-free fluorimetric analysis of the change in intrinsic protein fluorescence from aromatic tryptophan and tyrosine residues by using small quantities of solution. Thermal induced protein unfolding was monitored by detecting the emission shift at 330 nm and 350 nm at a laser power of 5%. The nanoDSF grade standard capillary chips (NanoTemper Technologies, München, Germany) were filled with 10 μL of freshly prepared (t0) formulation containing 25 mg/mL mAb compounded with either 0.01, 0.1, 1 or 10 mg/mL of the specific surfactant. Sodium dodecyl sulfate (SDS) was taken as positive control since the charge as been reported to lead to destabilization of proteins presumably by chare-charge interactions [55-57]. Samples were heated from 25 to 95° C. with a constant heating ramp of 0.5° C. per minute. The PR.StabilityAnalysis software (NanoTemper Technologies, München, Germany) automatically calculated the onset (Tₒₙ) and first transition point (Tₘ₁) temperature of the melting curve. Data reported are the mean value of three individual measurements.

### Evaluation of Protein Stability in Presence of Surfactants After Mechanical Stresses and Thermal Stability

Surfactant performance screens were carried out with 0.1 and 1 mg/mL of the surfactant in the formulation of the model mAb described within the Materials section. After compounding the liquid samples were sterile filtered through 0.22 μm Millex Sterivex™ GV (Millipore, Bedford, USA) filter units, filled into 6 mL type 1 glass vials and closed with Ø 20 mm teflon® coated serum stoppers (DAIKYO Seiko Ltd., Tokyo, Japan). The stoppered vials were crimped using an aluminum cap (Infochroma AG, Goldau, Switzerland). The corresponding placebo formulations were equally prepared, formulated and used as respective controls.

To evaluate the effects of the surfactants on the mAb's stability the formulations were exposed to various interfacial stress conditions like agitation and various freeze-thaw cycles. Shaking stress was carried by placing the vials horizontally for seven days at 5° C. and 25° C. protected from light within a shaker (HS 260 Control Model; IKA Werke GmbH & Co. KG; Staufen, Germany) at constant 200 rounds per minute (rpm). Freeze-thaw (F/T) stress was performed by exposing the vials to five consecutive cycles of freezing at −20° C. and thawing at 5° C.

Thermal stability data was generated by storing the liquid protein formulations for up to 24 months (mo) at 5° C., for 6 months at 25° C./60% relative humidity (rH) and 12 weeks at 40° C./75% rH. Samples were analyzed at initial time point (t0) and after 1 mo, 3 mo, 6 mo, 12 mo, 18 mo and 24 months of storage using the subsequently described analytical methods

### Visible Particles (VP)

Enhanced visual inspection was performed as previously described using a Seidenader V 90-T machine (Seidenader Maschinenbau GmbH, Markt Schwaben, Germany)[58]. The number of particles were categorized into four classes: class (I) is equivalent to 0 particles, class (II) is equivalent to 1-5 particles, class (III) is equivalent to 6-10 particles and class (IV) is equivalent to >10 particles.

### Turbidity (Opalescence and Clarity)

Turbidity was determined as previously described in literature and according to Ph. Eur. 2.2.1 using a 2100AN turbidimeter (Hach Lange GmbH, Dusseldorf, Germany) calibrated with a StablCal® calibration kit (Hach Lange GmbH). Results were given as Nephelometic Turbidity Units (NTU). [58, 59]

### Color

The color of the solutions was assessed by means of a LICO 690 Colorimeter (Hach Lange GmbH). Classification was performed according to the color scale described in Ph. Eur. 2.2.2. [60]. Data presented herein show the degree of color change according to color scale values of Ph.Eur. 2.2.2: class (I) is equivalent to color scale values of 9, 8, 7, or colorless; class (II) is equivalent to color scale values of 6 or 5; class (III) is equivalent to color scale values of 4 or 3; and class (IV) is equivalent to color scale values of 2 or 1.

### Light Obscuration

Sub-visible particle (SVP) count was measured by means of light obscuration using a HIAC 9703+ liquid particle counting system (Skan AG, Allschwil, Switzerland) and PharmSpec 3 (Hach Lange GmbH) software. The applied measurement technique was adapted from the method described in Ph.Eur. 2.9.19 [61] and USP <787>[62]. After rinsing the system with sample solution, four runs with a sample volume of 0.2 mL were performed. The final cumulative particle count was obtained by calculating the mean ±SD (standard deviation) from the last three measurements. SVP bigger than or equal to 2, 5, 10 and 25 μm were measured and presented as cumulative counts per mL of solution.

### Size-Exclusion High Performance Chromatography (SE-HPLC)

Detection of soluble mAb aggregates, in the following referred to as high molecular weight species (HMWS), the monomer and the low molecular weight species (LMWs) were analyzed by SE-HPLC. The utilized system comprised an Alliance 2695 HPLC instrument equipped with a 2487 UV detector (both from Waters Corporation, Milford, MA). The autosampler temperature was set to 5° C. and the system was loaded with total 100 μg of the mAb. Separation was performed using a TSK G3000 SWXL, 7.8×300 mm column (Tosoh Bioscience, Stuttgart, Germany) at a constant oven temperature of 25° C. and a mobile phase of 200 mM K₂HPO₄/KH₂PO₄ and 250 mM KCl pH 7.0 at a flow rate of 0.5 mL/min. Signal detection was executed at a wavelength of 280 nm and the Empower 3 Chromatography Data System software (Waters Corporation, Milford, MA) was used to calculate the peak area percent.

### Ion-Exchange High-Performance Liquid Chromatography (IE-HPLC)

Charge heterogeneity of the mAb was assessed by means of IE-HPLC using an Alliance e2695 HPLC instrument equipped with a 2489 UV detector (both from Waters Corporation, Milford, MA). The mAb was digested with carboxypeptidase and 50 μg were injected into a 4×250 mm ProPac™ WCX-10 (Thermo Fisher Scientific, Waltham, MA, USA) at a flow rate of 1.0 mL/min using a column temperature of 34° C. Elution of the mAb fragments was performed with solvents of increasing ionic strengths (mobile phase A: 20 mM MES, 1 mM Na-EDTA/mobile phase B: 250 mM NaCl, 20 mM MES, 1 mM Na-EDTA, pH 6.0). Signal detection occurred at a wavelength of 280 nm and Empower 3 Chromatography Data System software (Waters Corporation, Milford, MA) was used for data processing. The decrease of main peak was reported as percentage of total peak area (% area) over storage time.

### Surface Tension Measurement

Surface tension measurements were performed by means of liquid handling stations according to a method described by Amrhein et al.[63]. Briefly, the measurement depends on the correlation between drop mass and the surface tension of the sample. For this study a fully automated liquid handling station Freedom 384 EVO 200 (Tecan, Crailsheim, Germany) was equipped with an analytical balance (Mettler Toledo, Columbus, USA). The system contains stainless fixed tips which aspirate the sample with 100 μL·s⁻¹ from a sealed 1 mL round bottom LoBind® Deepwell Plate (Eppendorf, Hamburg, Germany). The sample was dispensed with 3 μL·s⁻¹ to a second round bottom Deepwell Plate 96/500 μL (Eppendorf, Hamburg, Germany) mounted on an analytical balance. The weight was recorded continuously using fully automated routines written in Matlab R2017b (MathWorks, Natick, MA, USA). The surface tension of water (72.6 mN/m) was used as a reference for calculations. The mean of three individual measurements of the samples (at t0) is reported as surface tension.

### Sub-Visible Particle (SVP) Counting by Backgrounded Membrane Imaging (BMI)

Sub-visible particle (SVP) count was measured by means of BMI on a Horizon instrument (Halo Labs, Philadelphia, PA) for the second set of proteins, mAb 2 and mAb 3. The system operates with polycarbonate 96-well membrane filter plates having 0.4 μm pores size (Halo Labs) that receives the sample for imaging. Under laminar flow, in each well of the membrane filter plate 50 μL of water for injection was added, plate vacuumed at 350 mbar and wells measured for background information. Subsequently, 40 μl of the sample was transferred to each well of the above plate, vacuumed at 350 mbar, washed with 50 μL of water for injection and vacuumed again at 350 mbar. The wells were finally measured and images analysis were performed in the Horizon Vue software. Particle count is reported as an average of three measurements and a maximum of 6.4% of the filter plate coverage.

### Example 1

### 1.) Measurement of Surfactant Stability Against Enzymatic Ester Hydrolysis

Since polysorbate hydrolysis due to small amounts of co-purified host cell proteins represent a major challenge for long-term protein formulation stability, an assay was developed to test for the ester stability in presence of two model lipases, PCL and CALB, at 0.25 and 0.5 mg/mL. FIG. 2 shows a representative chromatogram of PS20 before (solid line) and after enzymatic digestion (dashed line).

TABLE 2
Retention times of the hydrophilic (1) and lipophilic (2) part
of tested surfactants. Results are given as the average of three
individual measurements with a standard deviation of ≤0.15.

Retention time (min)

| | hydrophilic | lipophilic |
|---|---|---|
| Surfactant | non-esterified peak (1) | ester main peak (2) |
| PS 20 | 7.1 | 13.4 |
| HS15 | 6.9 | 13.6 |
| RH 40 | 7.8 | 13.4 |
| Chol-PEG | 12.3 | 17.8 |
| TPGS | 7.5 | 18.2 |

Before digestion, the hydrophilic fraction is eluted at lower retentions times between 7-8 min (Table 2) and increases with increasing size of the average polyethylene oxide subunits in the following order: HS15 (15 PEO units)<PS20 (20 PEO units)<TPGS (23 PEO units)<RH40 (40 PEO units)<Chol-PEG (45 PEO units). The broader peak shape of fraction (1) might be explained by the polymeric character of PEO part and the associated size distribution of the different polymeric chains. Due to the chemically more heterogeneous composition PS20, HS15 and RH40 due to the degree of esterification (presence of mono-, di- and partially tri- and tetra-esters), [24] the lipophilic part fraction (2) eluted as multiple subsequent peaks, whereas for TPGS and Chol-PEG only one peak was obtained (data not shown). It seems that for the latter two surfactants the lipophilic past is chemically more homogeneous.

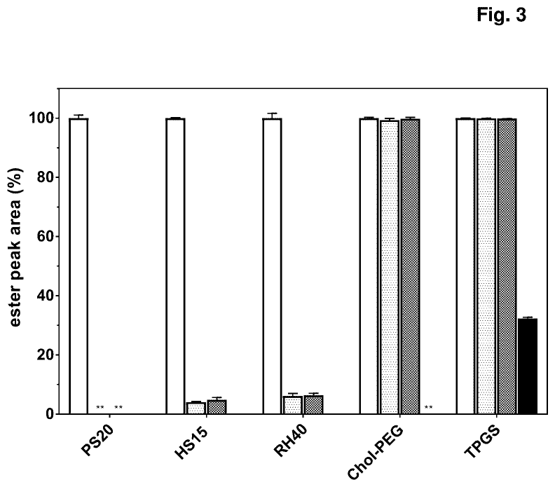

After incubation with the lipase mixtures, the chromatogram obtained for PS20 shows an increase in hydrophilic fraction (1) and a complete loss of the lipophilic fraction (2) (FIG. 2 dashed line), which clearly shows the cleavage of all the ester bonds. FIG. 3 shows the degree of surfactant degradation due to enzymatic hydrolysis with 0.25 and 0.5 mg/mL lipase mixtures. For PS20, HS15 and RH40 a strong enzymatic hydrolysis was observed (>95%). No difference in the degree of degradation between the two tested lipase concentrations was observed. In contrast, TPGS and Chol-PEG did show only a negligible enzymatic hydrolysis for both lipases mixtures tested (<0.3%). To exclude a “false positive” result of this test, a chemical hydrolysis with 0.1 mmol NaOH was performed. Under these conditions, ester bonds of Chol-PEG were completely hydrolyzed (100%), shown by disappearance of fraction (2), whereas TPGS was only partially hydrolyzed (68% of fraction (2) remaining).

The data showed that HS15 and RH40 surfactants resulted into a comparable degradation as PS20 during the experiment. Since the enzymatic ester hydrolysis of polysorbates is a major challenge within protein formulations surfactants with a comparable degradation to PS20 were in subsequent studies.

### Evaluation of Thermal Conformational Protein Stability in Presence of Surfactants

Maximization of the conformational stability is reported to increase long-term drug product quality and/or stability by preventing unfolding and aggregation of therapeutic proteins. High throughput & low volume screening techniques are DSC (Differential Scanning Calorimetry) or measuring the intrinsic protein fluorescence under isothermal chemical denaturation (ICD) or thermal denaturation conditions by means of nanoDSF (Differential Scanning Fluorimetry) [49]. To exclude a negative impact of the surfactants on the protein's conformational stability thermal DSF measurements were performed. The distinct conformation of the multi-domain structure of mAbs, including CH2, CH3 and Fab, is crucial for their binding ability and therapeutic efficacy. For mAbs, an independent and stepwise unfolding of specific domains is presumed, with CH2 being generally least stable followed by Fab and CH3 [48].

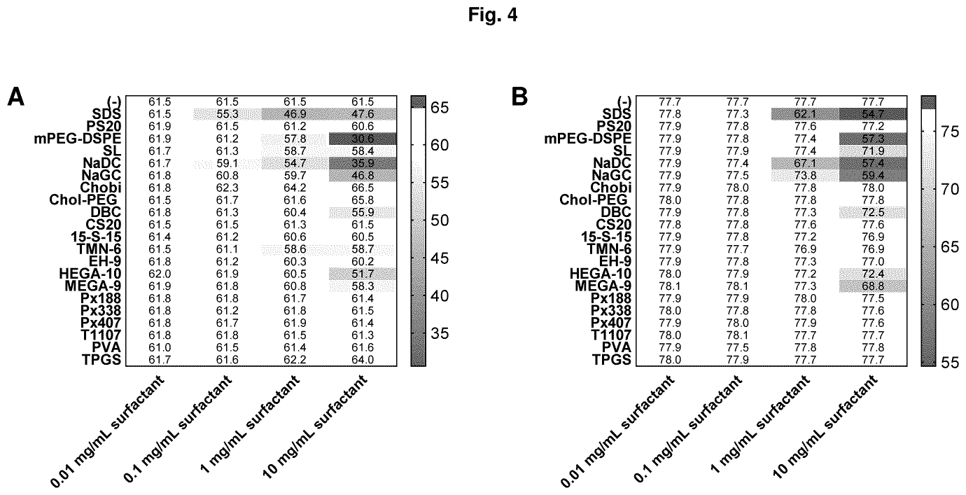

Stability indicating parameters being the onset temperature (Tₒₙ) of unfolding and the first melting transition (Tₘ₁) were measured for the model mAb in presence of surfactants at concentrations ranging from 0.01 mg/mL to 10 mg/mL (see FIG. 4). The values of the mAb without added surfactant (Tₒₙ=61.5±0.3, Tₘ₁=77.7±0.1) were taken as reference and the data was presented as a heat map: the darker the color, the stronger is the decrease in the respective temperature due to the presence of the surfactant. In general, the majority of the conditions tested did not show any significant effect on the mAbs' conformational stability within therapeutically relevant surfactant concentrations.

As expected, SDS showed a concentration dependent destabilizing effect as compared to the reference formulation without any surfactant; the higher the concentration the bigger the decrease in both Tₒₙ and Tₘ₁. SDS, known to be a potent destabilizer of protein's conformational stability, was used as positive control [55-57]. Similar to SDS, a strong decrease in thermal conformational stability properties was observed for surfactants possessing a negative charge like NaDC, NaGC and mPEG-DSPE already at lower concentrations of 0.1 & mg/mL. However, for positively charged molecules like T1107 the conformational stability parameters remained unaffected. Since the model mAb with an isoelectric point (pI) of 8.7 possessed an overall positive net charge at the chosen formulation conditions of pH 7.0 a charge-charge interaction between mAb and negative charged surfactant can be assumed resulting into the observed different behavior of charged surfactants. Another interesting finding was a slight increase of Tₒₙ, after addition of non-ionic sterol-(Chol-PEG and Chobi) or vitamin E-based (TPGS) surfactants at concentrations of 1 mg/mL or higher (Chobi and TPGS) and 10 mg/mL (Chol-PEG). A potential interference of fluorescence signals was excluded since the surfactant stock solutions were measured and revealed no considerable fluorescence signals. Therefore, a slight stabilizing effect of these surfactants of the mAbs' native state is more likely. In contrast, DBC also a non-ionic sterol based surfactant showed a slight decrease in Tₒₙ, (1 and 10 mg/mL) and Tₘ₁ (10 mg/mL). This phenomenon might be explained by structural properties different in either sterol group modifications or the presence of glucamide functions as follows: (i) DBC contains a free hydroxyl group at position 3 of the sterol structure (similar to NaDC and NaGC), while Chol-PEG and Chobi hold sterically larger functional groups at this position, (ii) DBS contains a sterically large hydrophilic gluconamide functionalization at position 20 of the sterol structure, while for Chobi and Chol-PEG the original hydrophobic cholesterol-structure at this position remained unaltered. The latter might be supported by the comparable destabilizing effect on the Tₒₙ (1 and 10 mg/mL) and Tₘ₁ (10 mg/mL) comparing DBC, MEGA-9 and HEGA-10, which all have a gluconamide-functionalization as hydrophilic moiety in common.

Comparing the impact of all surfactants containing open vs. closed sugar ring structures revealed a destabilizing effect on the mAbs' conformational stability for DBC, MEGA-9 and HEGA-10, and SL at elevated concentrations while Chobi showed no impact indicating an independence of an open-vs.-closed sugar conformation.

Comparison of the alcohol ethoxylates revealed no conformational destabilizing effects for CS20 at all concentrations tested. Slight decreases in the Tₒₙ (1 and 10 mg/mL) were observed in the following order: 15-S-15=EH-9<TMN-6 while all showed similar decreases in Tₘ₁ at 10 mg/mL. The observed differences might be explained from a structural perspective as follows: (i) linear (CS20) vs. branched alcohol ethoxylates (15-S-15, EH-9, TMN-6), respectively, primary vs. secondary alcohol ethoxylates, or (ii) decreasing number of the average PEO-subunits (CS20 (20-24 PEO-subunits)>15-S-15 (15 PEO-subunits)>EH-9 (9 PEO-subunits)>TMN-6 (6 PEO-subunits). However, CS20 is the only alcohol ethoxylate that was available in pharmaceutical grade quality, therefore, a higher purity may be assumed as compared to the 3 other molecules. Remaining impurities like free alkyl residues might also result into conformational destabilizing effects, a phenomenon well known for polysorbates.

A subset of the surfactants screened, including a few that showed impact on the conformational stability, were taken into a next screen to evaluate their impact on the mAb's stability during long-term studies but also after mechanical and thermal stresses. To clarify the predictive character of this high throughput screening (HTS) some of the surfactants with potential liabilities were additionally included in the following surfactant performance screen: mPEG-DSPE, SL and DBC.

### Example 2: Alternative Surfactant Performance Screen

### 1. Evaluation of Protein Stability in Presence of Surfactants After Mechanical Stresses and Thermal Stability

The impact of mechanical/interfacial stresses on the mAbs' stability was tested performing agitation and freeze-thaw studies. Additionally, the thermal stability of the formulations was studied. The data collected for tested attributes like visible particles (VP), color and turbidity was classified into 4 categories that allows for presentation as a heat map. Formulations with more unfavorable attributes like many VPs, strong color changes or high turbidity were categorized into higher classes and marked in different grey intensities (darker intensity equals to stronger change in parameter).

Surfactant levels studied were kept constant at 0.1 and 1 mg/mL. For the ease of reading, the surfactants were classified based upon their hydrophobic moiety into 4 sub-groups being: (i) acyl-based, (ii) alkyl-based, (iii) sterol-based, and (iv) others. Formulations containing either no surfactant (see FIG. 11), PS20 (see FIG. 11) or Px188 (see FIG. 14) were used as guiding references to assess the performance of the alternative surfactants.

### Acyl Group

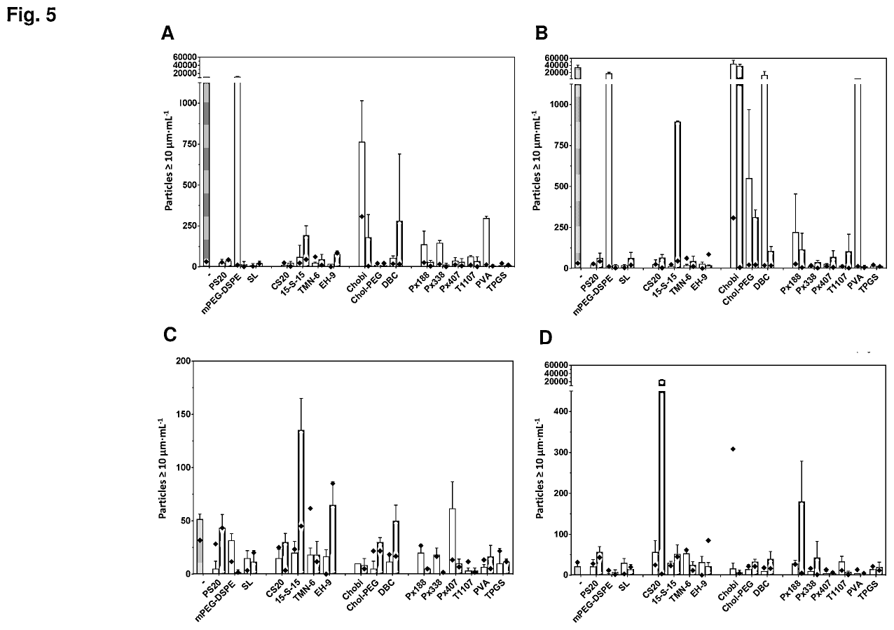

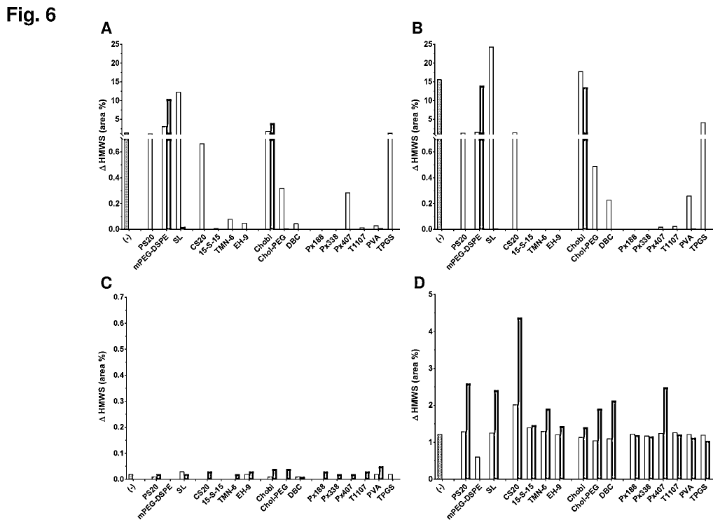

In the acyl group the mPEG-DSPE showed a remarkably poor outcome with many VP and high turbidity values, which were not seen in placebo formulations. Since this phenomenon was mainly seen for the 1 mg/mL formulation a reason could be the already described charge-charge interaction which reduces mAbs conformational stability (FIG. 4). Additionally, mPEG-DSPE possess a comparably high DST like seen for the sterol based surfactants. These findings were unexpected since mPEG-DSPE has a low critical micelle concentration (CMC: 1×10⁻⁶ M) and a comparably small flexible structure. Without being bound to theory, an explanation could be a charge-charge interaction of mPEG-DSPE and mAb resulting in a higher apparent molecular weight with lower diffusivity with low amounts of surfactant molecules being present at the interface. Moreover, a slow disintegration of the mPEG-DSPE micelles could also be involved leading to diminished interfacial stability properties. In contrast, SL showed better results during most tests and the outcome in terms of VP, turbidity and DST (FIG. 11) was comparable to PS20. SL showed considerably enhanced stability at 1 mg/mL presumably upon shaking, here the change in HMWS of >10% was considerably increased at lower surfactant concentration (FIG. 6). In the pre-screens, both SL and mPEG-DSPE showed conformational destabilizing effects at higher concentrations, but no or only marginal effects at the lower concentration (FIGS. 4 and 5). To verify the findings from the pre-screens as a predictive measure they were compared to the outcome of the thermal stress test. In the case of mPEG-DSPE the outcome appears to support these findings. For this surfactant only the 1 mg/mL, not the 0.1 mg/mL, formulation showed high amounts of SVP and an increased turbidity.

Low amounts of visible particles were found in the control formulation with PS20. Both concentrations, but especially 0.1 mg/mL PS20 showed good stabilizing effects during all applied stresses. In contrast, Px188 formulations contained many large protein particles upon most stress tests particularly during shaking at the lower surfactant concentration. Comparing the dynamic surface tension (DST) of both compounds, a remarkable difference was observed at 1 mg/mL, but not at lower surfactant concentration (FIG. 11).

### Alkyl Group

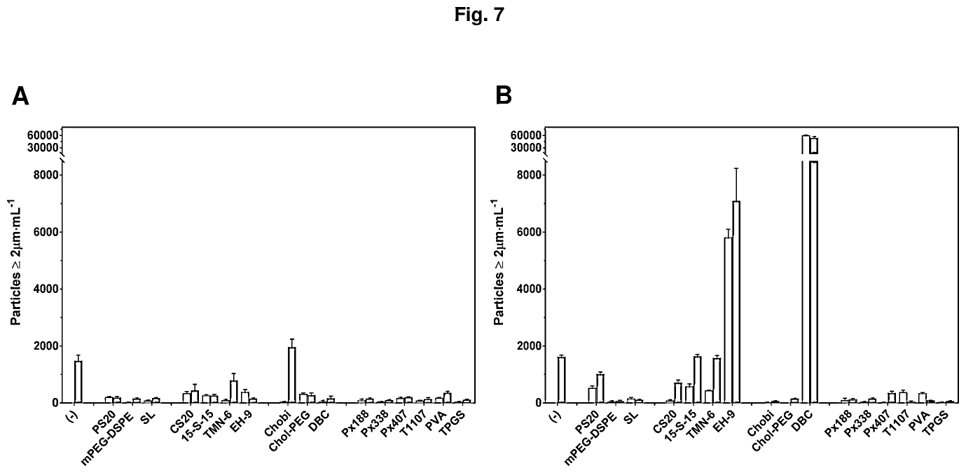

Another group phenomenon was seen for the alkyl based surfactants with higher amounts of VP and turbidity (FIG. 12) especially at high concentrations. These particles were often also observed in placebo formulations and appeared pronounced in EH-9 formulations with high amounts of SVP≥2 μm (FIG. 7). The presence of insoluble impurities is likely and may account for VPs in this group, but was not further investigated. An exception was CS20 the only surfactant of this group available in pharmaceutical grade. Here both concentrations tested were without visible particles at the initial time point. Similar findings were obtained for 15-S-15. The 1 mg/mL formulation of CS20 showed increased degradation at elevated temperature conditions, accompanied by an increase in VP and turbidity (FIG. 12). Additionally, an increase in SVP (FIG. 7) and HMWS (FIG. 6D) was observed. The degradation theory was supported by 1H-NMR measurements (data not shown). TMN-6 performed well, showing no substantial impact to protein quality attributes during stress testing or long-term storage. Increased turbidity was observed for the higher (1 mg/mL) formulation. Compared to other surfactant groups the alkyl based compounds comprised the lowest DST which might be explained by the flexible structure of these surfactants leading to a high packing density at interfaces. Nevertheless, solubility issue makes it hard to construe the outcome of the stress tests and further investigation is needed to identify potential impurities and their influence. Thus, the performance evaluation of these molecules as alternative surfactants would be facilitated. In summary, 15-S-15 and TMN-6 all showed acceptable quality (especially for the 0.1 mg/mL formulations) after applied interfacial and thermal stresses, which in some cases were even slightly superior to PS20.

### Sterol Group

Interestingly, most sterol based surfactants showed no or negligible stabilizing effects. After shaking stress, these formulations showed comparably high amounts of VP, a high turbidity and in the case of Chobi even a strong change in color. The presence of insoluble impurities is likely and may account for VPs in this group, but was not further investigated. The strongest particle formation upon shaking was seen for Chobi. This formulation even behaves similar to formulations without surfactant. Moreover, both formulations have a similar dynamic surface tension of around 73 mN/m. The DST describes how effective a surfactant can disturb cohesive forces within the interface and how strong it can adapt to changes at the interfaces e.g. during shaking. The high DST of both Chobi and control formulation indicates less protein stabilizing effects at the interface and might again explain the outcome of the shaking studies. The missing stabilizing effect at the interface is also seen upon F/T studies for both, Chobi and control formulation without surfactant. In general, most surfactants showed good stabilizing effects with little amounts of VP upon freeze-thaw stresses. The data suggest some correlation between DST and outcome of the shaking studies with increasing VP for higher DST values. Since other factors like surfactant/impurity solubility or surfactant-protein interactions may also influence VP formation, further investigation is needed. Besides Chobi, also Chol-PEG and DBC showed a particle formation particularly after agitation and comparably high DST values. However, Chol-PEG resulted in good protein quality attributes (i.e. HMWS and loss of IE-HPLC main peak) in all test conditions, especially at the higher 1 mg/mL formulation condition. Although visible particles were observed after shake stress testing, the test conditions are far harsher than observed in reality and the results were comparable to the Px188 control. Furthermore, the set-up with 1 mg/mL DBC already showed many particles initially (FIG. 13) and in placebo formulation indicating solubility issues. Despite VP also SVP>10 μm (FIG. 5) and soluble aggregates (FIG. 6) were increased after shaking especially when performed at 25° C. Moreover, Chobi and DBC exceeded USP <787> criteria of maximum 6,000 particles≥10 μm per container. In contrast F/T and thermal stress did not show any considerable increase in HMWS or SVP.

Previous investigations on sterol based surfactants reported a prolonged adaption of the rigid and bulky sterol ring structure to form favorable conformations at the interface. The surfactant alignment is more complicated for a larger surfactant with a rigid backbone and therefore a longer time (>2 h) to reach equilibrium surface tension was reported [64, 65]. This data supports the outcome of our shaking experiments. Sterol-based surfactants may not react quickly to changes at the interface, e.g. during shaking.

### Others

This group includes the polymeric surfactants, poloxamer, poloxamine and polyvinyl alcohol as well as the tocopherol based compound TPGS. All formulations within this group showed lower amounts of VP and turbidity values (FIG. 14) at the higher surfactant concentration. Upon shaking stress formulations with 1 mg/mL also showed considerable lower amounts of SVP (FIG. 5) and soluble aggregates (FIG. 6). Interestingly the strongest particle formation was seen for Px188. Data suggests a dependence of the HLB of poloxamers and poloxamine on the ability to protect mAb against interfacial stresses. Compared to Px188 and Px338 with relatively high HLB>27 the outcome of the stress tests for Px407 (HLB: 22) and T1107 (HLB: 18-23) was considerable better, already at a concentration of 0.1 mg/mL. Because of the compounds different HLB and molecular weights slightly different dynamic surface tensions were expected. However, the commercial samples are heterogeneous compositions of molecule mixtures with averaged molecular masses and HLBs which might explain the outcome of the DST measurements. In general, the DST of the surfactants of this group were comparably high and might explain the less stabilizing effect especially at lower surfactant concentration. Comparing the molar ratios (mAb:surfactant) with PS20 reference formulation it should be noted that the polymeric surfactants have an approximately 10-fold lower molar ratio of around 1:0.004 (mol/mol) for 0.1 mg/mL concentration, respectively 1:0.04 (mol/mol). These findings could also account for the less stabilizing effects upon shaking.

Rather unexpected was the good quality of formulations with 1 mg/mL TPGS. The vitamin E based surfactant TPGS also possess a rigid ring structure similar to the sterol-based surfactants, but with an additionally attached alkyl chain. Moreover, it also showed a rather high DST which was also almost constant for both concentrations already seen for the sterol-based surfactants.

Therefore, comparable results after shaking stresses were expected, but interestingly 1 mg/mL TPGS performed very well in terms of VP, SVP and HMWS. It seems that measuring the DST not always gives reliable predictions for the tendency of particle formation upon shaking and further investigation is needed.

In summary, formulations with 1 mg/mL SL, T1107, Px338, and Px407 were of acceptable quality upon all applied stresses and storage conditions. Moreover, at this concentration, formulations with PVA and TPGS showed very good stabilizing effects with superior product quality. Even though the DST with ˜60 mN/m was comparably high these compounds are promising alternative surfactant candidates. 15-S-15 and TMN-6 showed acceptable quality upon all applied stresses and storage conditions at lower surfactant concentrations (0.1 mg/mL).

### 2.) Long-Term Protein Stability

Under long-term storage conditions a potential negative impact on protein stability should have been excluded. Therefore, stability of the formulations was evaluated for 6 months storage at 5° C. and 25° C. in terms of formation of visible and sub-visible particles, changes in color and turbidity as well as monomer content (FIG. 11-14).

The formulation without surfactant was used as reference additionally the alternative surfactant formulations were compared to the established surfactants PS20 and Px188 known to lack a negative influence. Reference formulation without surfactant showed high amounts of VP after 6 months' storage at both temperatures. Additionally, the SVP content ≥10 μm at 25° C. storage was slightly increased compared to initial. Comparable results were found for PS20. Here, the formation of VP was observed especially at higher concentration and at elevated temperature, but SVP amount was not increased (FIG. 11). In contrast to the results of the stress studies formulations with a concentration of 0.1 mg/mL of Px188, long-term storage showed good stability at both temperatures during all analytics. Grapentin et al. showed comparable stabilizing results of PS20 and Px188 in liquid mAb vial formulations upon long term storage [66].

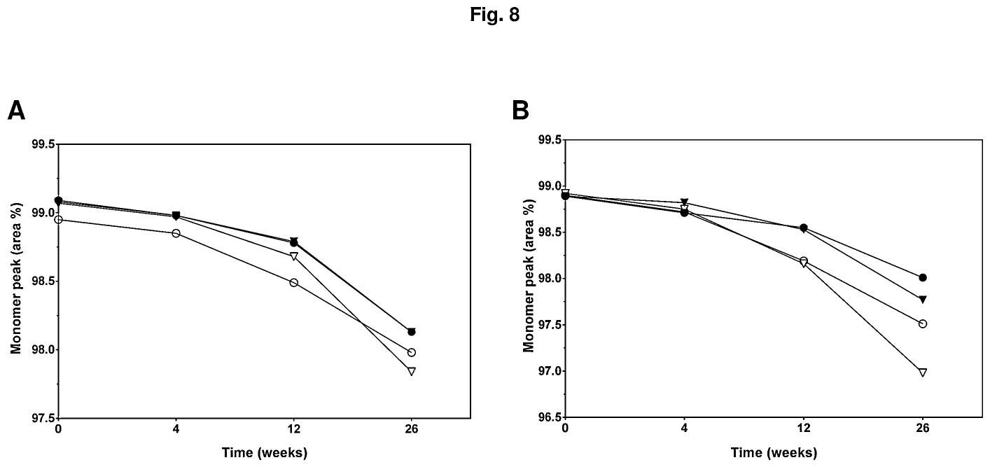

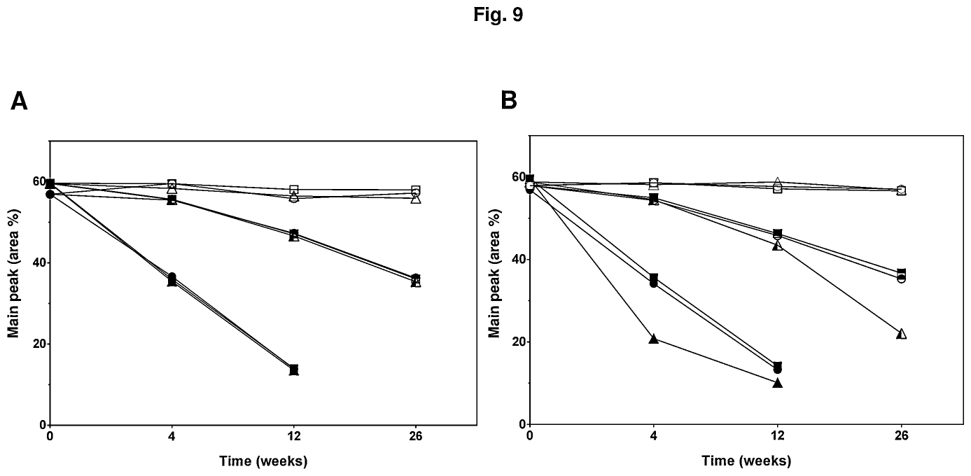

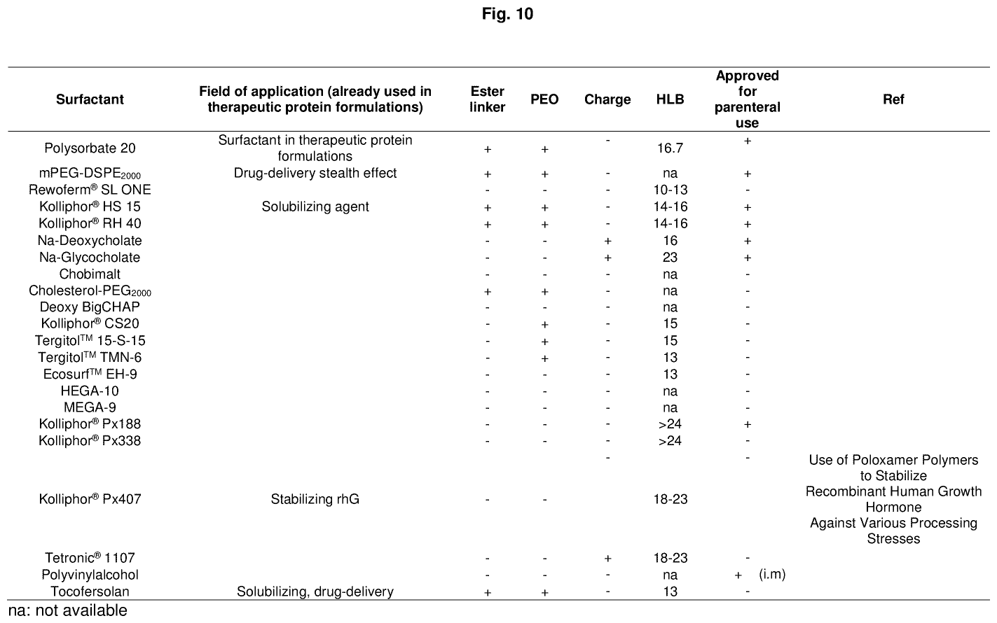

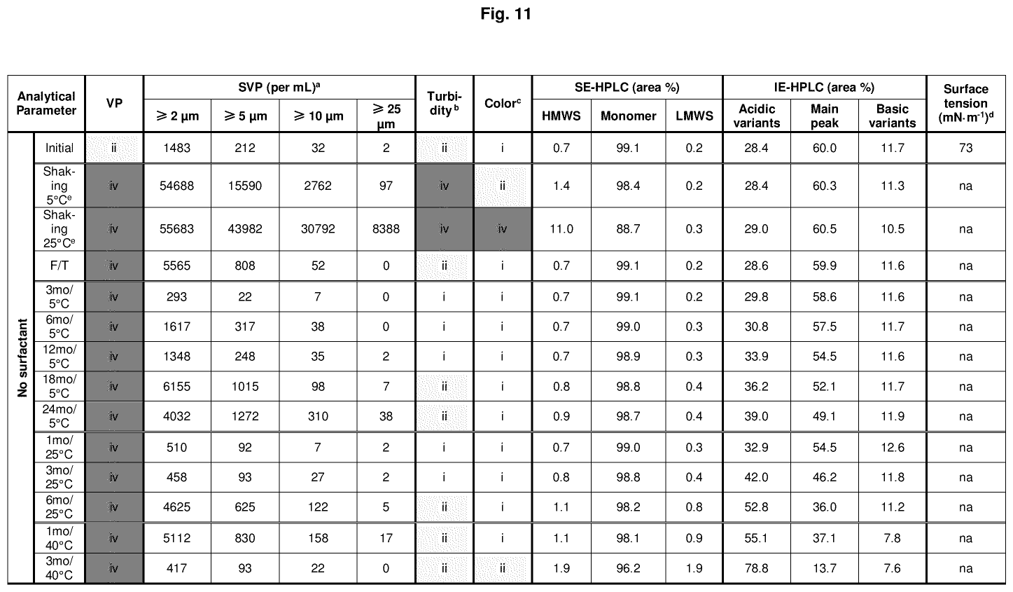

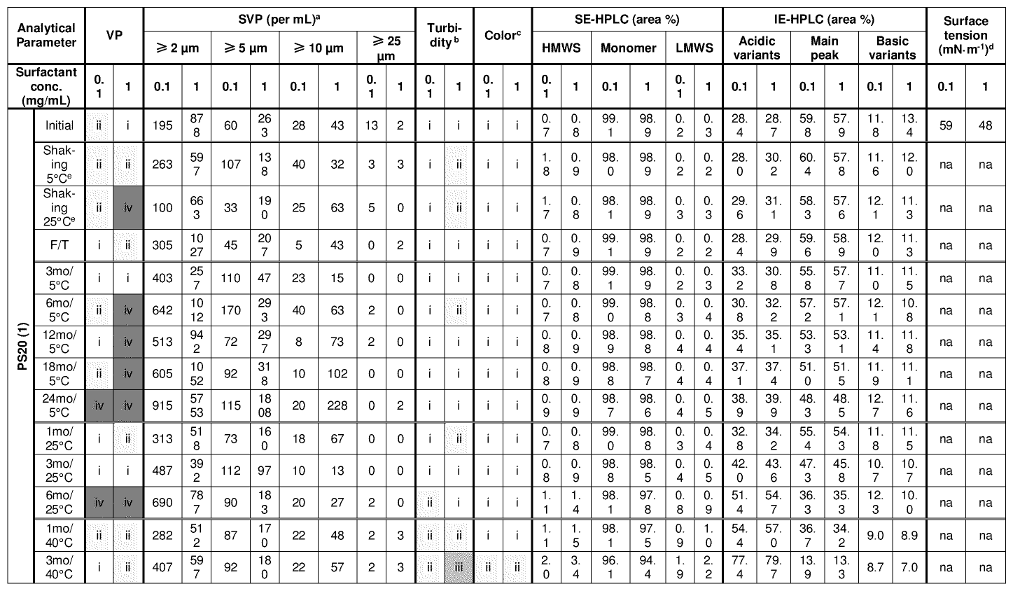

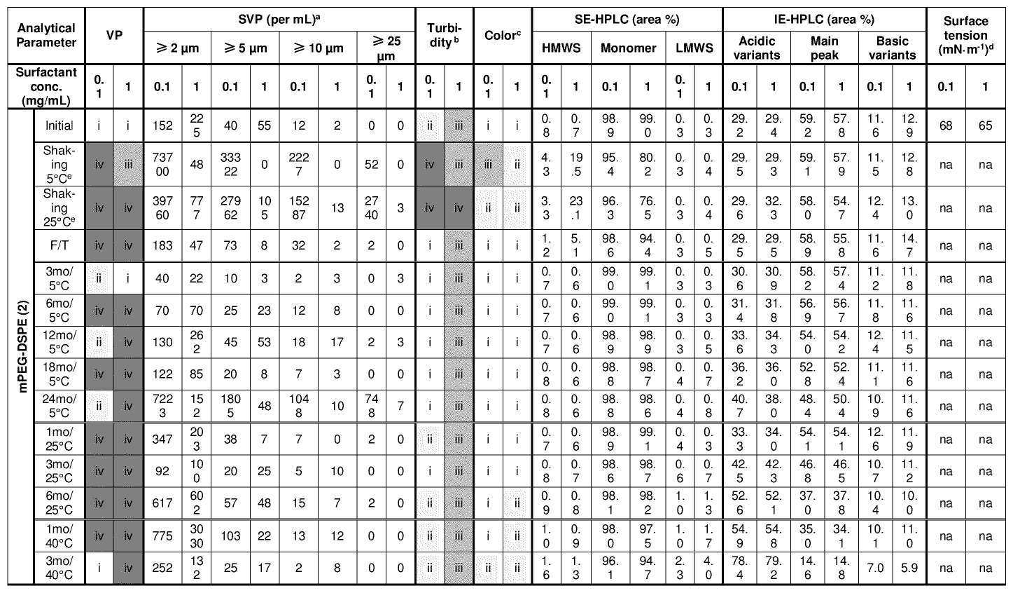

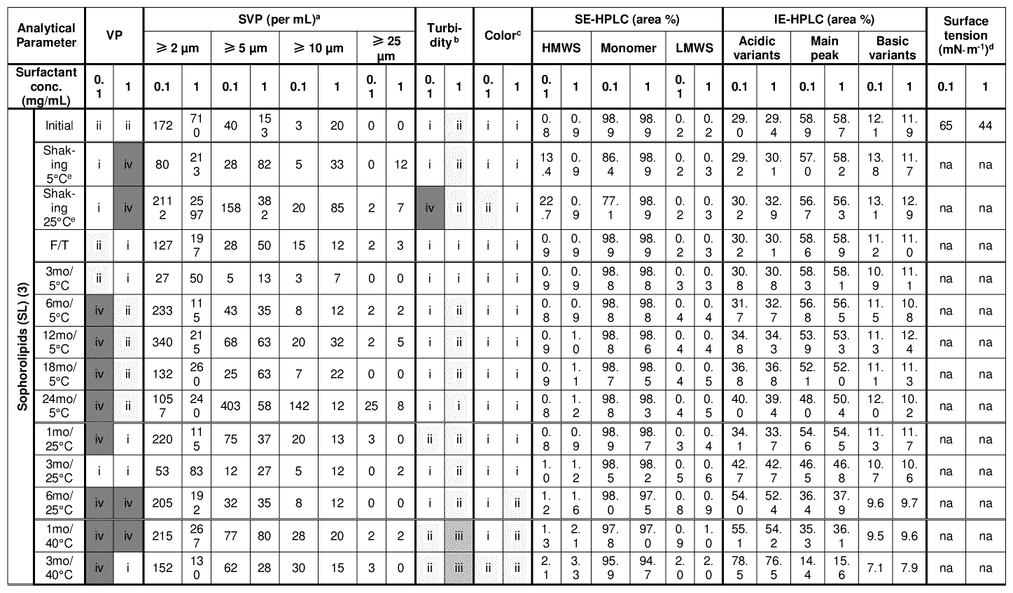

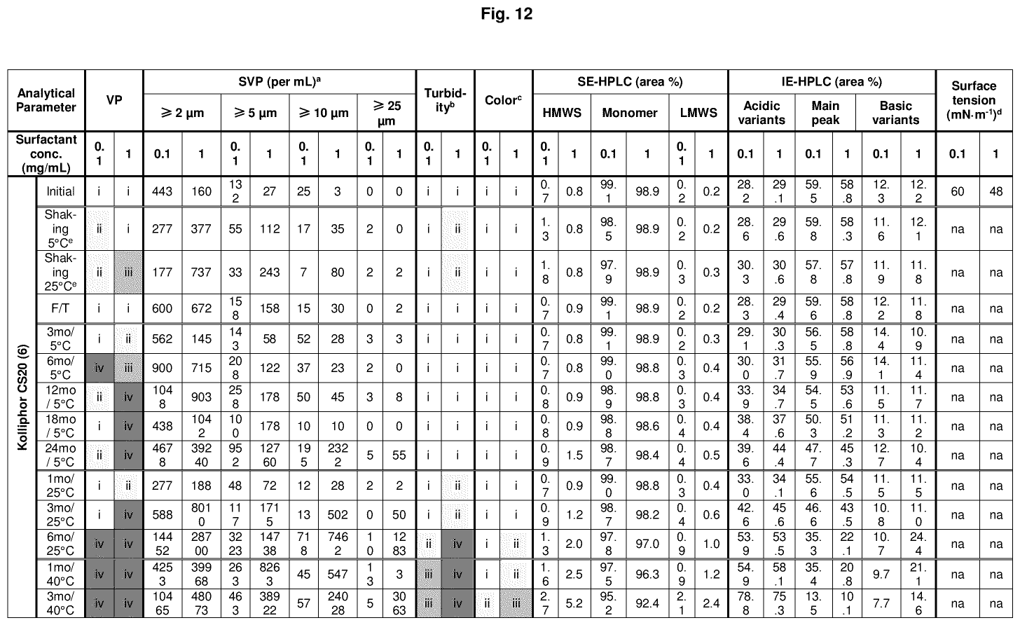

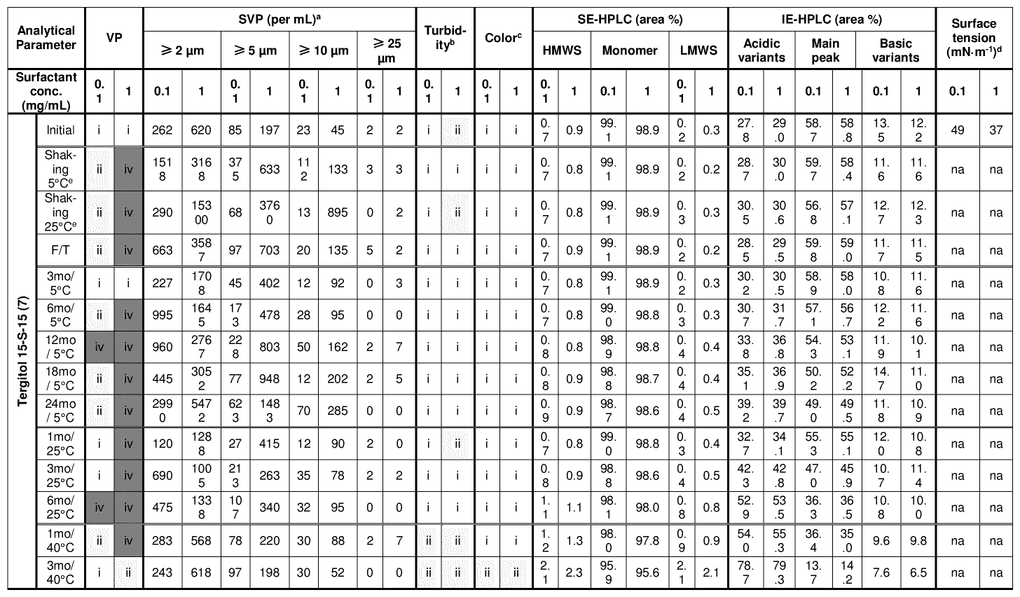

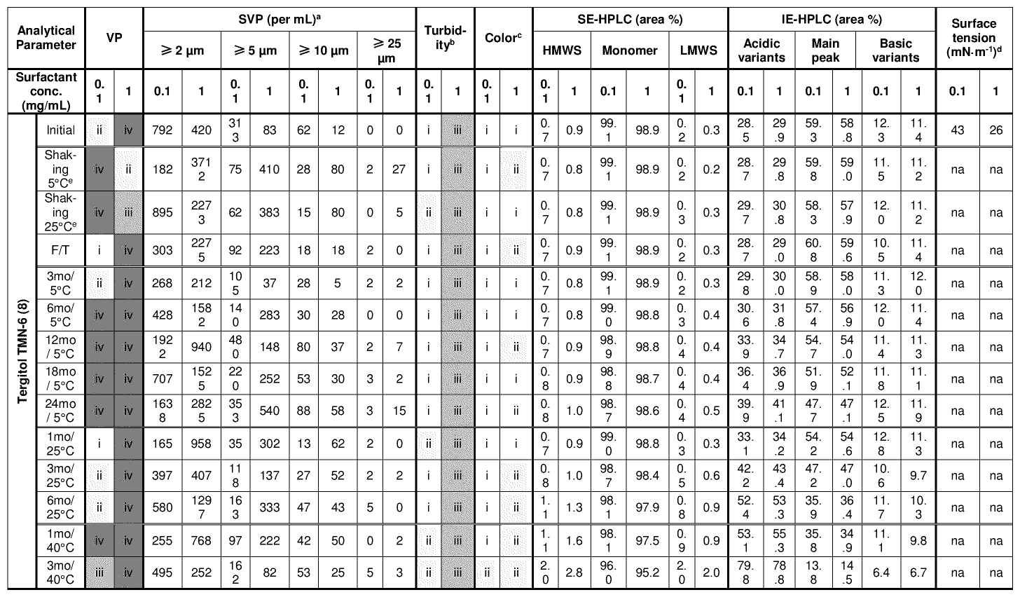

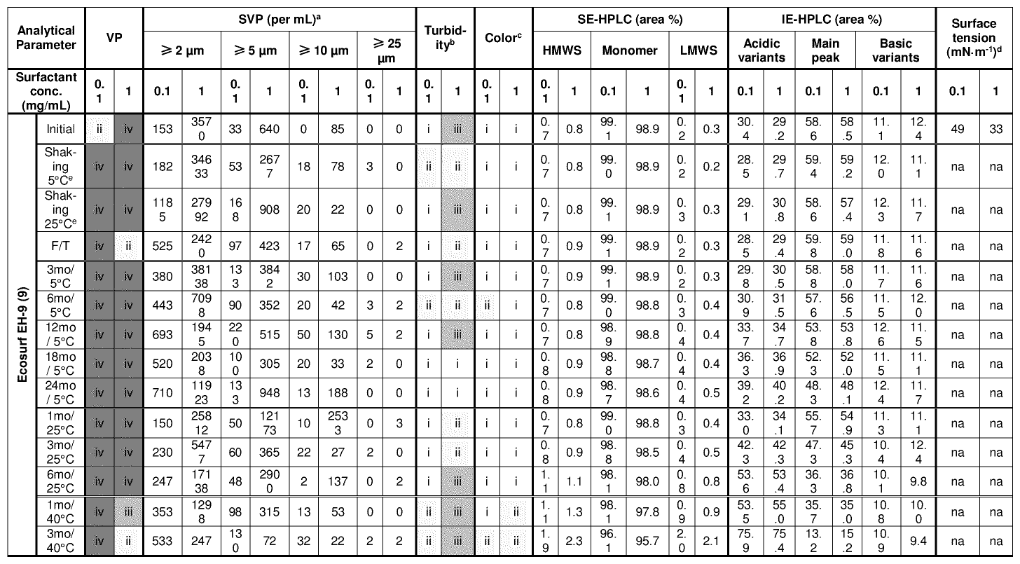

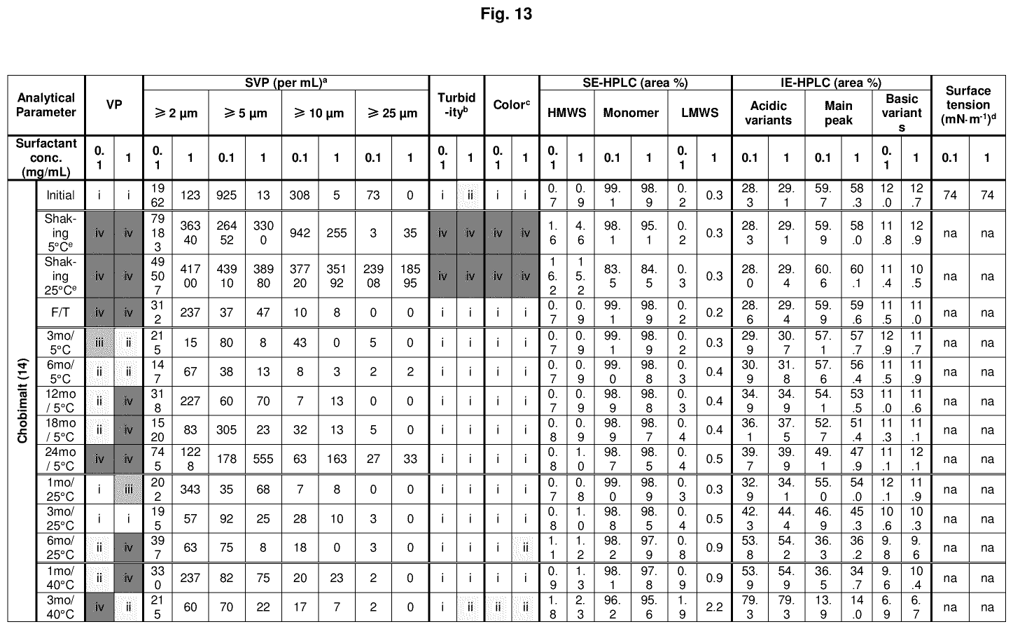

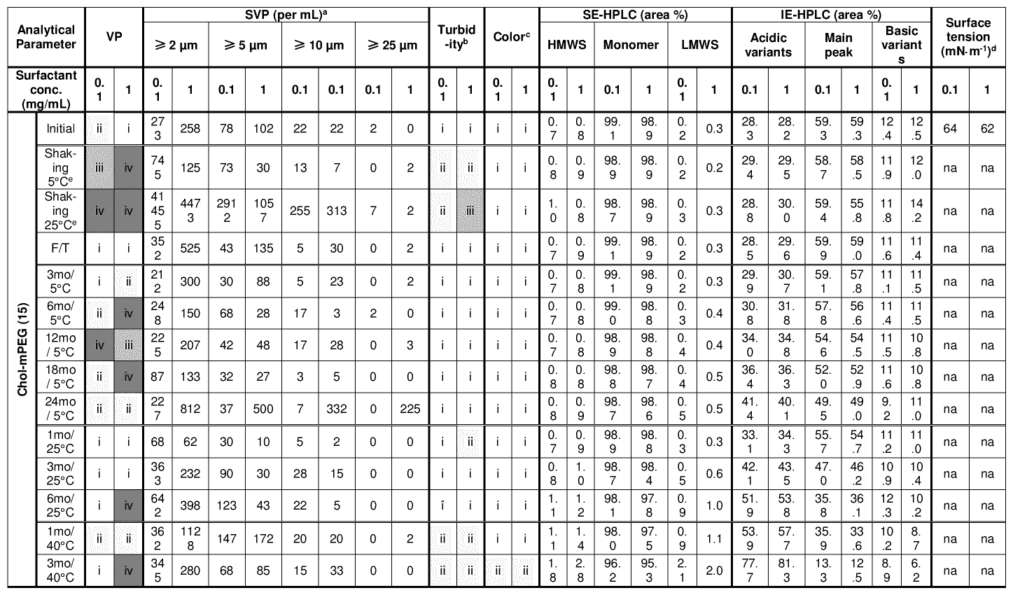

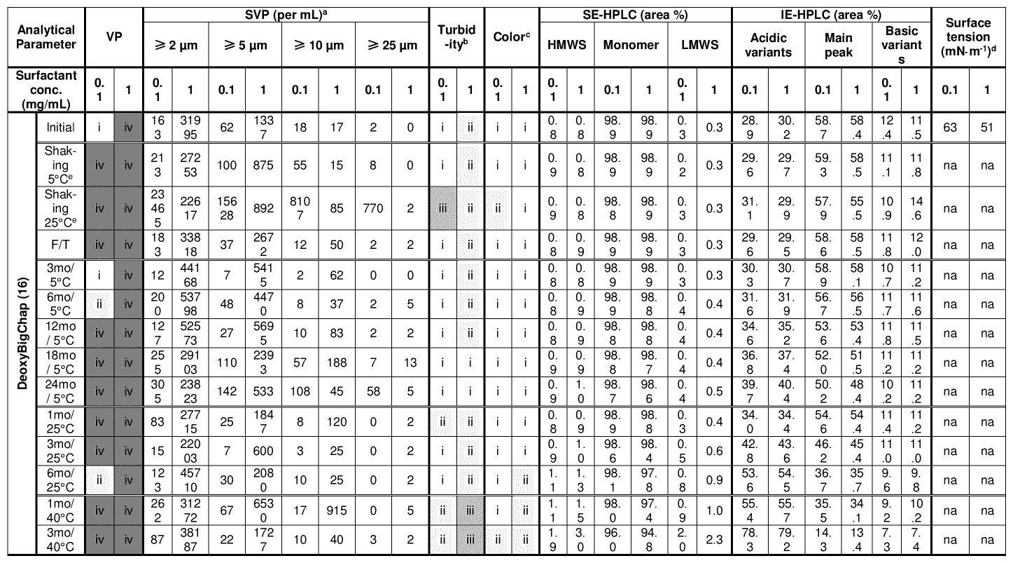

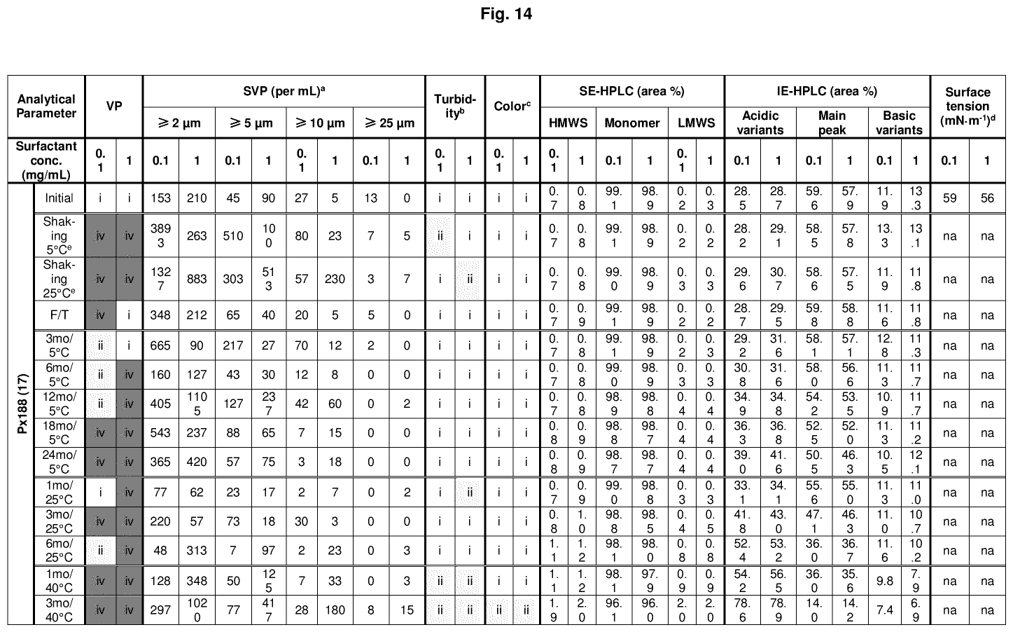

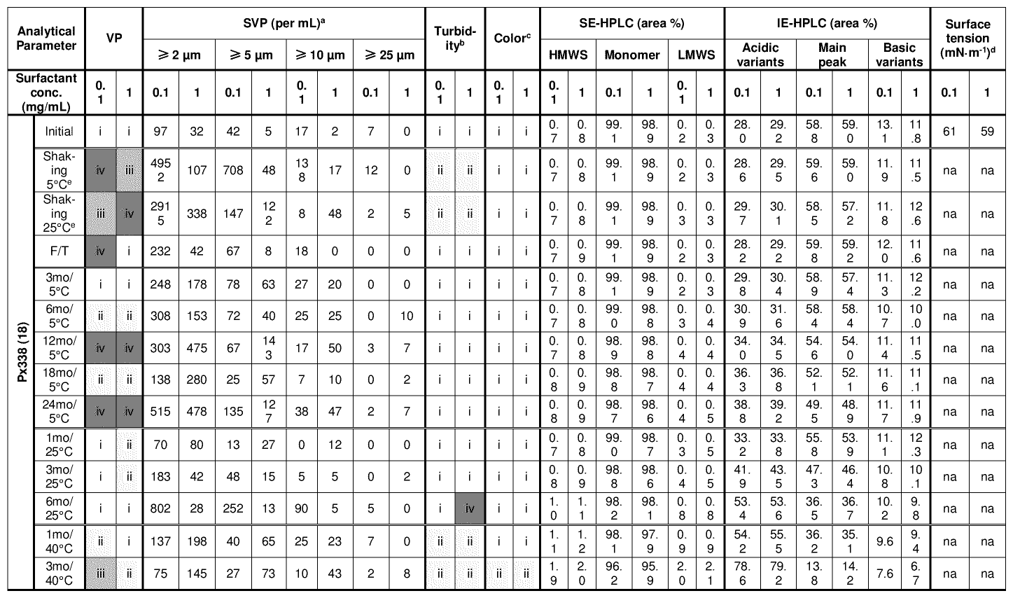

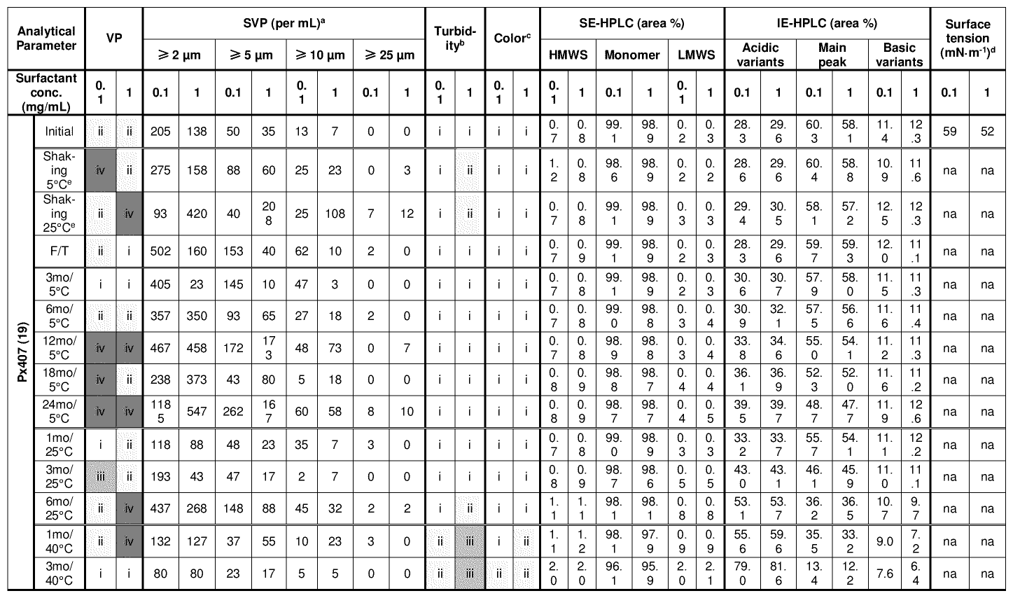

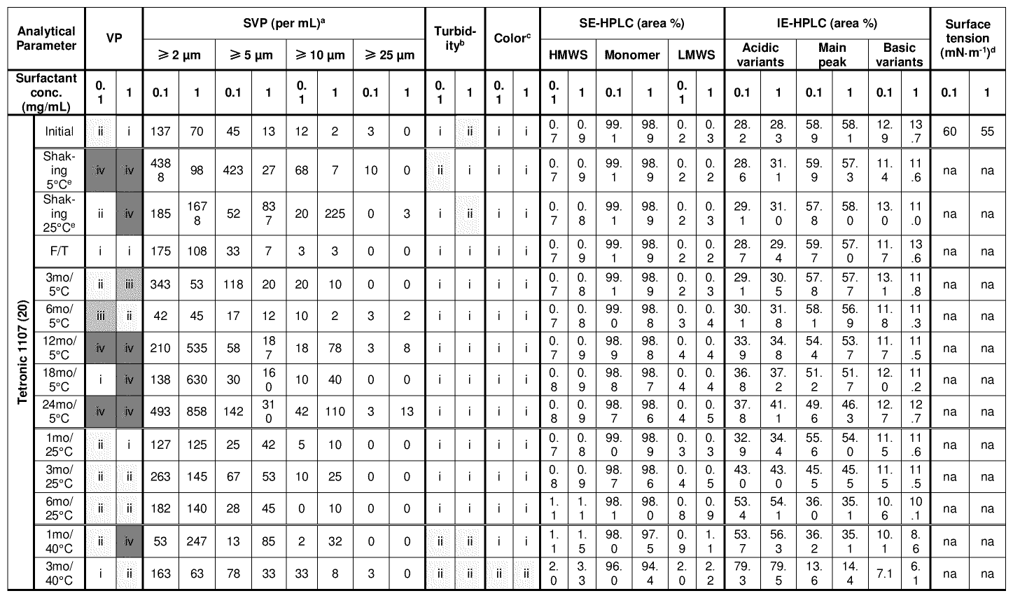

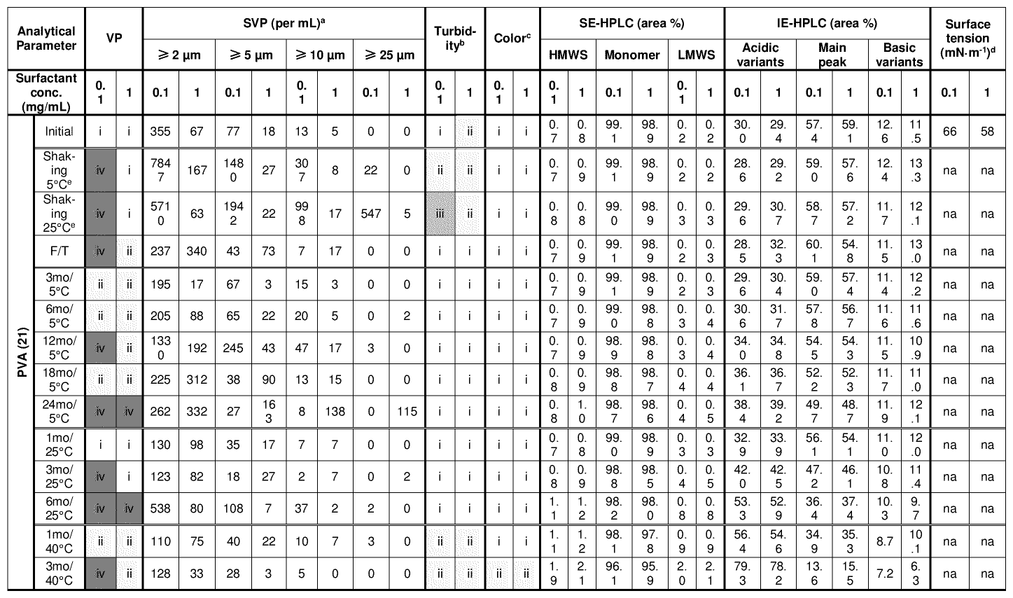

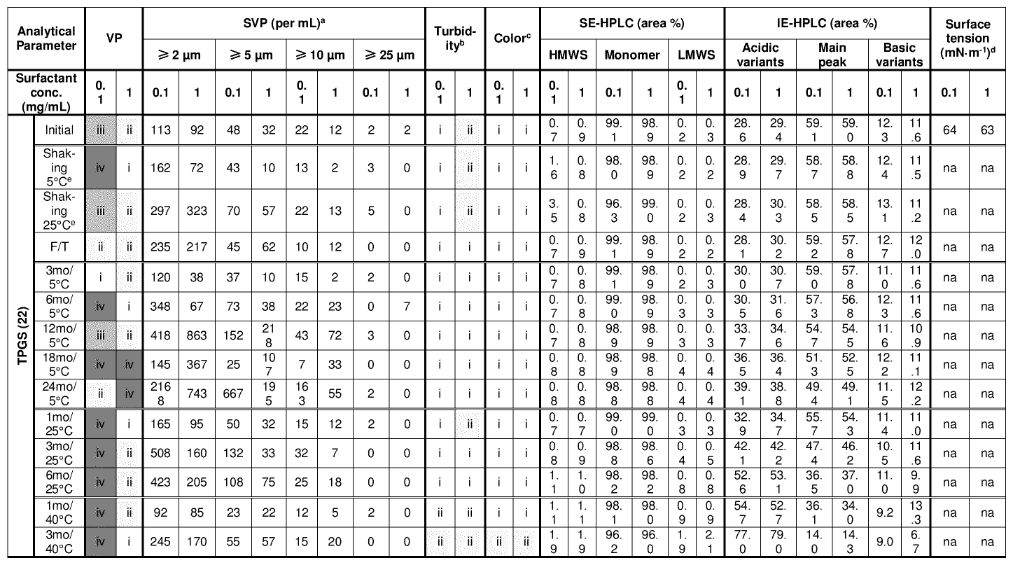

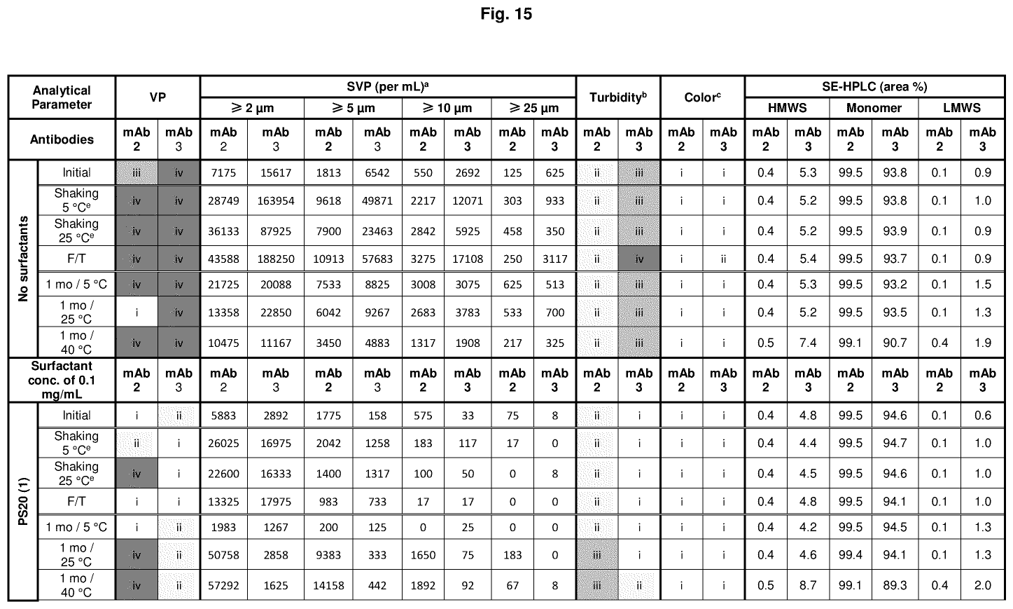

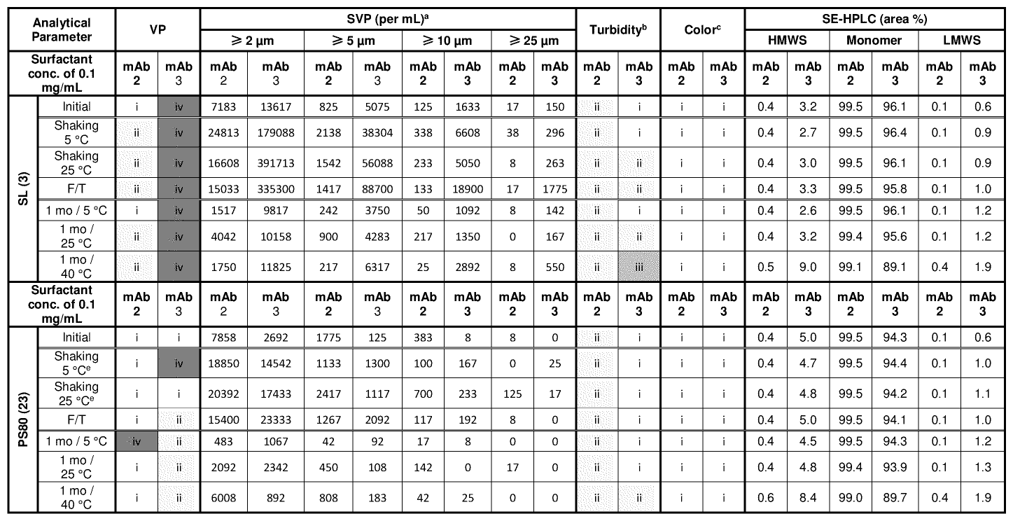

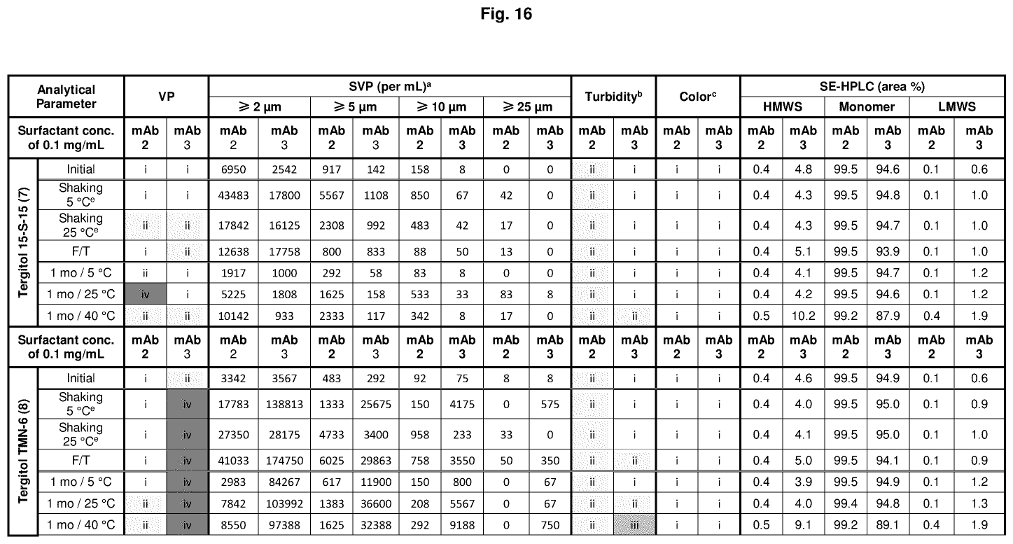

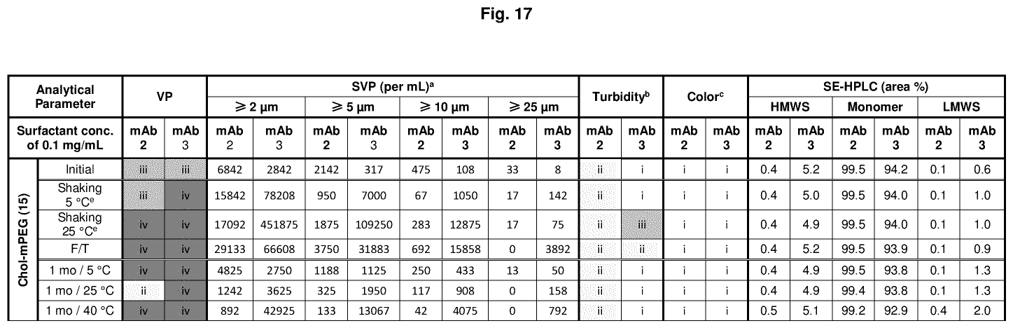

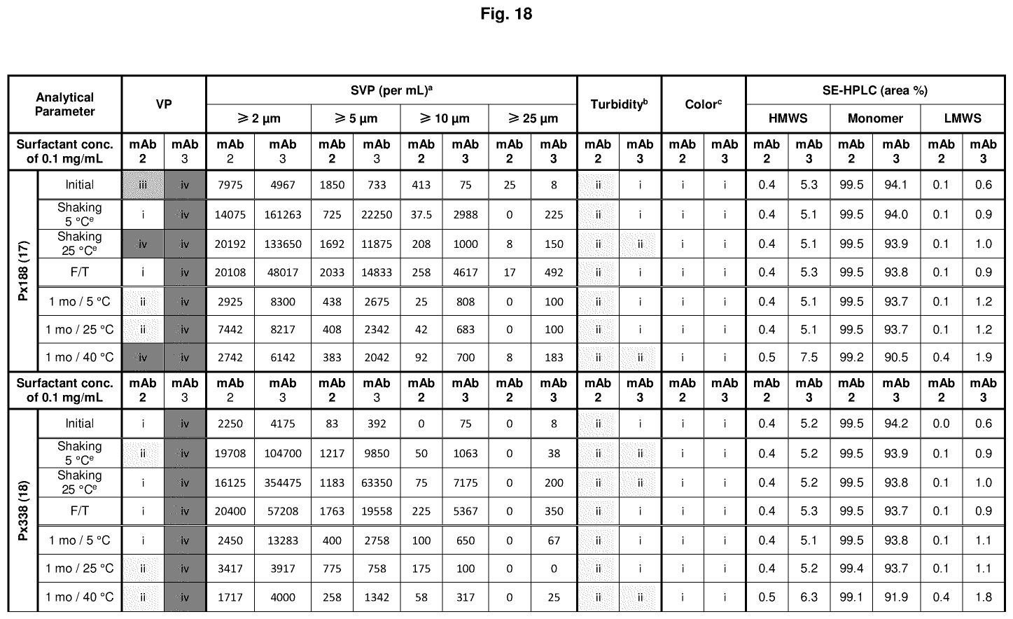

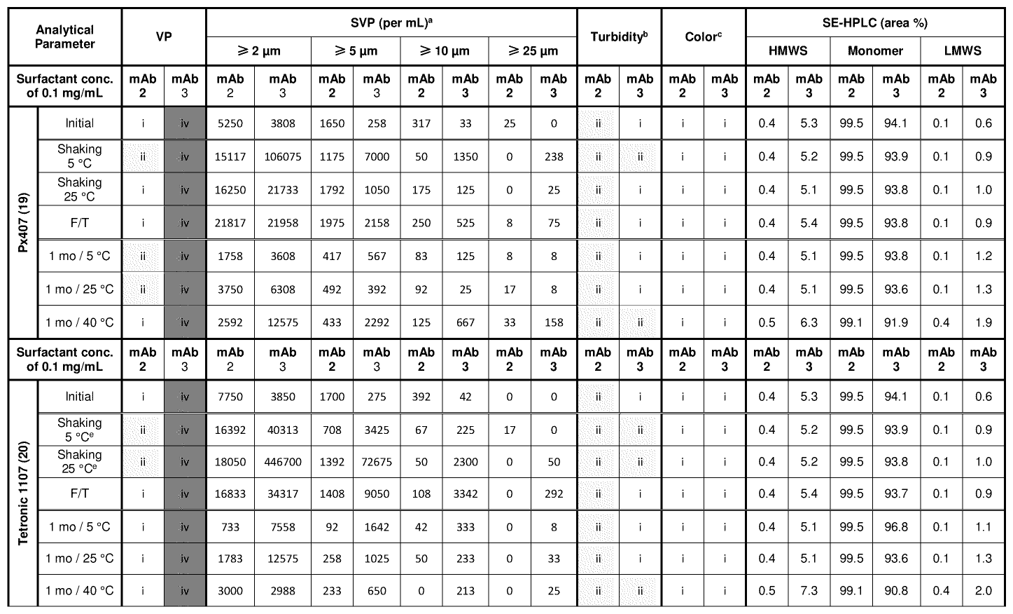

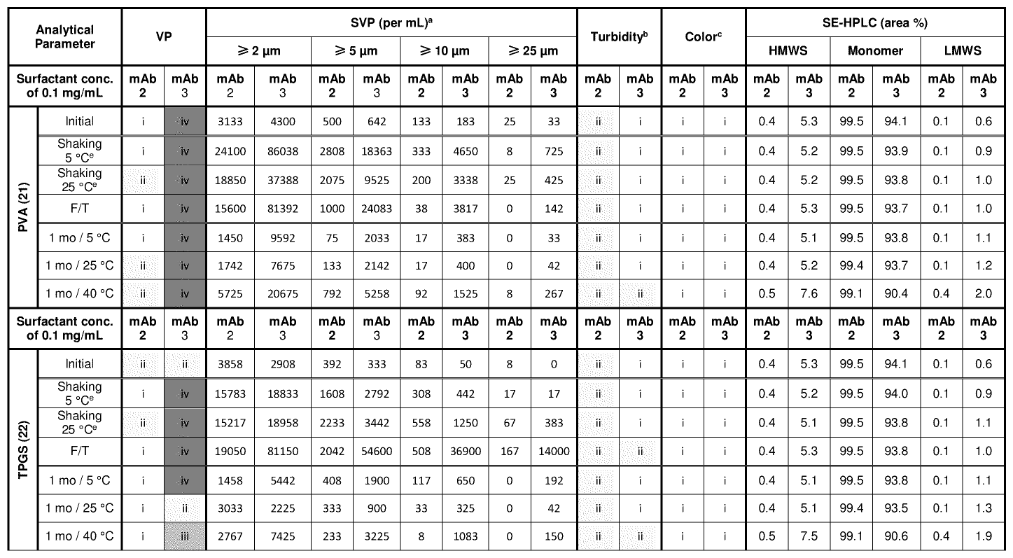

All three reference formulations did not show any changes in color, turbidity and monomer content (FIGS. 11 and 12). However, it has to be noted that most formulations did not show any considerable changes during these tests. Sub-visible particle counts measured by light obscuration are in general at low levels and the reported values are significantly lower than the maximum numbers accepted according to pharmacopoeia USP <787> and Ph.Eur. 2.9.19. A slight increase in SVP levels was observed for CS20 formulations at elevated temperatures which might be explained by the previously described thermal degradation. Additional to the SVPs a higher turbidity and a decrease in monomer content by means of SE-HPLC was observed (FIG. 8). Moreover, IE-HPLC of CS20 formulations revealed a decrease of the main peak area compared to reference formulations with PS20 and Px188 at elevated stress temperature conditions (FIG. 9). This may suggest degradation of the mAb initiated by CS20 degradation products.

An increased turbidity was also observed for the higher concentration of the alkyl surfactants TMN-6 and EH-9. The beforehand described phenomenon might be due to solubility problems of potential impurities or the surfactants itself. Due to their small and flexible structure one can assume a good performance upon interfacial stresses for these surfactants but also a stronger tendency for oxidative degradation as already described for PS20 [25, 26]. Unfortunately, the solubility issue especially at higher concentration makes the interpretation of the performance difficult from a particle perspective.

The present studies could not confirm a correlation between long term stability and conformational stability. VP amount of mPEG-DSPE and SL formulations was high at both concentrations but only SL and not mPEG-DSPE showed a slight decrease of monomer content at higher concentration (FIG. 8). The evaluation of formulations with DBC, the third surfactant which showed slight decreases in conformational stability, was also critical since higher concentrations revealed solubility issues. In summary, the slight changes in Tₒₙ observed in the pre-screening (FIG. 4) in our case do not have a predictive character on the protein stability so far.

In general, the surfactants in the “others” group showed good results upon long term storage with low VP and SVP as well as a high monomer content. The exception are PVA formulations where VP formation was observed at elevated temperatures at both concentrations but not at 5° C. Further time points have to be analyzed to make statements on the performance of PVA as alternative surfactant at ambient storage temperatures of 2-8° C.

In contrast to the outcome of the interfacial stress tests the stabilizing properties of sterol based surfactants upon long term storage especially at low concentrations was very good. It seems this surfactant group needs more time to adsorb at the interface and therefore may not protect proteins as well upon fast interface changes present during shaking, or require higher concentrations to adequately stabilize. However, when the surfactant has sufficient time to adsorb at the interface it can evolve its stabilizing effects. Nevertheless, a drug product (DP) will be exposed to mechanical stresses during transportation hence, most of the sterol based surfactants tested are not suitable as an alternate to the established surfactants PS20 and Px188.

### 3) Evaluation of Protein Stability for mAb 2 and mAb 3 in Presence of a Surfactants Selection After Mechanical Stresses and Thermal Stability

We further investigate a selected panel of surfactants (PS20, PS80, SL, 15-S-15, TMN-6, Chol-PEG, Px188, Px338, Px407, T1107, PVA and TPGS) regarding their efficiency to stabilize protein formulations. The lower level of 0.1 mg/mL surfactant in presence of two different mAbs (2 and 3) at a concentration of respectively 25 mg/mL and 10 mg/mL was tested. PS80 was added to include one supplementary control to the PS20 (FIG. 15) and Px188 (FIG. 18) surfactants. This supplementary study was setup to confirm the positive effect of the promising surfactant candidates from the first screen (i.e. for mAb1 herein) on the stability of two other mAbs under mechanical and thermal stress. The conditions are identical to the ones previously described above: shaking at 200 rpm for 7 days at 5° C. and 25° C., 5 freeze/thaw cycles of −20° C. to 5° C. and storage for 4 weeks at 5° C., 25° C. and 40° C.

While the PS20 and PS80 protects the protein well in mechanical stress conditions such as shaking and F/T especially in the case of mAb 3, the storage at elevated temperature of PS20 formulations for 4 weeks yields high counts of subvisible particles in the case of mAb 2 (FIG. 15).

In the alkyl surfactants group, especially 15-S-15, display a good protection in these mechanical stress conditions for mAb 2 and mAb 3 with lower risk of degradation by HCPs (FIG. 16). 15-S-15 was able to show comparable or superior protein protection compared to the polysorbates PS20 and PS80 as well as Px188 for three different mAbs including a bispecific antibody-fragment with known lower conformational protein stability (mAb 3). These findings make 15-S-15 a very promising candidate as an alternative surfactant. TMN-6, another surfactant tested out of the alkyl class, showed good protection for mAb 2, while many VP were observed after mechanical and thermal stress for mAb 3.

Compared to PS20 and PS80, the surfactants SL, PVA, the poloxamers Px338 and Px407, T1107 and TPGS did not perform as well under shaking conditions, but compared to Px188 the protection of mAb 2 was better with less VP, especially at 25° C. (FIG. 15 and FIG. 18). For mAb 3, no differences of VP count between these surfactants and Px188 could be observed upon shaking as they all showed rather high counts. However, the VP count for storage of these surfactants with mAb 3 over 4 weeks is in general lower compared to Px188. In the case of Chol-PEG, the above trends of superior protection compare to Px188 were not observed (FIG. 17). The tendency for the SVP is slightly different, with all surfactants and mAb 2 formulations being in the same range when mAb 3 formulations are having dramatic differences in SVP counts between some surfactants, especially in the shaking conditions. Moreover, for mAb 3 in shaking conditions, we observe that PVA, Px407, T1107 and TPGS displays lower counts than Px188, with TPGS even being in the close range to PS20 and PS80 (FIG. 15 and FIG. 18).

In terms of soluble mAb aggregates, most of the conditions and surfactants displayed the same amounts of HMWS for mAb 2 and only two cases with major differences for mAb 3. The storage at 40° C. for mAb 3 shows that Chol-PEG, Px338, Px407, T1107, PVA and TPGS result in less HMWS than the polysorbates (FIG. 17 and FIG. 18), and SL has a surprising effect on soluble aggregates with the overall lowest HMWS amount for mAb 3, but still has the presence of high SVP counts (FIG. 15).

As a general outcome from this selection of surfactant candidates, proteins and conditions we can identify 15-S-15 as one of the most efficient surfactants as it is equivalent and even better than PS20, PS80 and Px188 in the wide range of formulations and for all mAbs tested. TPGS, Px338, Px407, PVA, T1107, TMN-6 and SL were less protective during mechanical stress compared to the polysorbates, especially with mAb 3 but the performance was equivalent or better than Px188. Moreover, the surfactants Px338, Px407, PVA, T1107 and TPGS were tested for mAb 2 and mAb 3 at a lower surfactant concentration than the ideal range seen in the first screen for mAb 1 but still displayed a better protective effect than Px188. In the cases of 15-S-15 and TMN-6, the ideal range of concentration seen for mAb 1 was also used for the testing of mAb 2 and mAb 3, in which both surfactants retained their overall protective effect. The positive performance of Chol-PEG from the first testing (i.e. with mAb 1) was not confirmed with mAb 2 and mAb 3. Indeed, in this second study the protective effect of mAb 2 and mAb 3 was lower compared to Px188 in most conditions (FIG. 17).

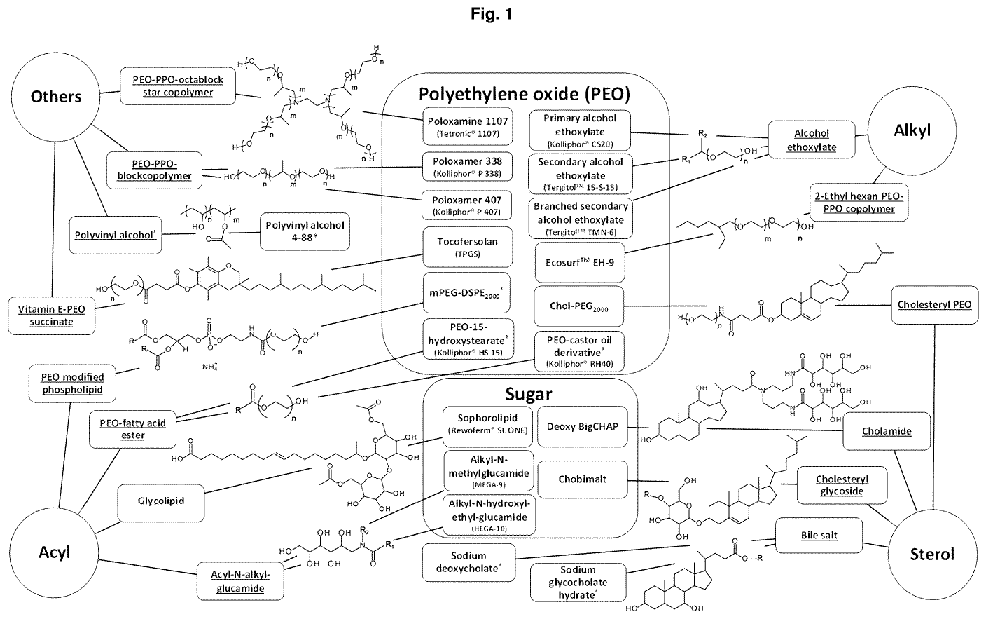

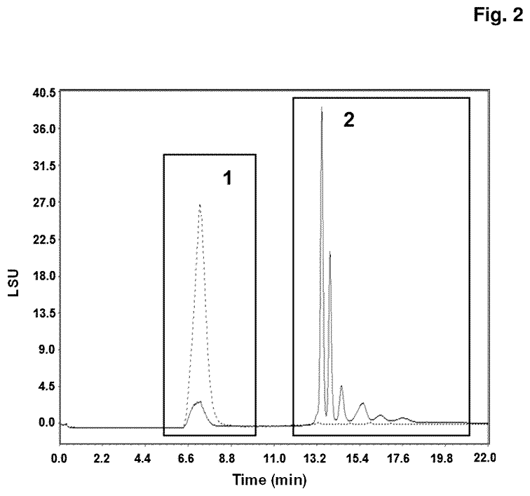

In summary, the present invention did not identify a positive effect on stability of protein formulations, based on an entire surfactant class or subgroup as e.g. shown in FIG. 1. Surprisingly, with TPGS, PVA, T1107, Px338, Px407, TMN-6, 15-S-15, Chol-PEG, and SL the present invention identified nine surfactants showing comparable or superior protein stabilizing effects than the established PS20, PS80 and Px188. In particular, TPGS and PVA at 1 mg/mL showed very good stabilizing properties during both interfacial and thermal stress conditions with low amounts of VP, SVP and HMWS. However, compared to the traditional surfactant Px188, also formulations containing 1 mg/mL Px338, Px407, T1107, Chol-PEG and SL showed superior stabilizing effects with lower amounts of VP and SVP in most applied stresses and during stability. Although SL formulations showed a slight decrease of the monomer content after long-term storage at elevated temperatures, these surfactants are regarded as potential alternatives to PS20 and Px188 since intended storage conditions are 2-8° C. For 15-S-15 and TMN-6 formulations, even lower surfactant concentrations were sufficient to provide good protein stabilizing properties comparable to PS20. Again, also in the presence of mAb2 and mAb3, lower concentrations of 0.1 mg/mL 15-S-15 provides the best stabilizing properties of all surfactants, even surpassing PS20 and PS80. TPGS, PVA, Px338, Px407, TMN-6, SL, T1107 are better or comparable to Px188.

### References

[1] M. S. Kinch, An overview of FDA-approved biologics medicines, Drug Discov Today, 20 (2015) 393-398.

[2] A. Mullard, 2017 FDA drug approvals, Nature Reviews Drug Discovery, 17 (2018) 81.

[3] H. C. Mahler, W. Friess, U. Grauschopf, S. Kiese, Protein aggregation: pathways, induction factors and analysis, J Pharm Sci, 98 (2009) 2909-2934.

[4] T. A. Khan, H. C. Mahler, R. S. Kishore, Key interactions of surfactants in therapeutic protein formulations: A review, Eur J Pharm Biopharm, 97 (2015) 60-67.

[5] H. C. Mahler, F. Huber, R. S. Kishore, J. Reindl, P. Ruckert, R. Muller, Adsorption behavior of a surfactant and a monoclonal antibody to sterilizing-grade filters, J Pharm Sci, 99 (2010) 2620-2627.

[6] T. Rubic-Schneider, M. Kuwana, B. Christen, M. Assenmacher, O. Hainzl, F. Zimmermann, R. Fischer, V. Koppenburg, S. D. Chibout, T. M. Wright, A. Seidl, M. Kammuller, T-cell assays confirm immunogenicity of tungsten-induced erythropoietin aggregates associated with pure red cell aplasia, Blood Adv, 1 (2017) 367-379.

[7] Y. Gallais, N. Szely, F. X. Legrand, A. Leroy, M. Pallardy, I. Turbica, Effect of growth hormone and IgG aggregates on dendritic cells activation and T-cells polarization, Immunol Cell Biol, 95 (2017) 306-315.

[8] J. Carpenter, B. Cherney, A. Lubinecki, S. Ma, E. Marszal, A. Mire-Sluis, T. Nikolai, J. Novak, J. Ragheb, J. Simak, Meeting report on protein particles and immunogenicity of therapeutic proteins: filling in the gaps in risk evaluation and mitigation, Biologicals, 38 (2010) 602-611.

[9] W. Wang, S. Nema, D. Teagarden, Protein aggregation-pathways and influencing factors, Int J Pharm, 390 (2010) 89-99.

[10] M. C. Manning, J. Liu, T. Li, R. E. Holcomb, Rational Design of Liquid Formulations of Proteins, in: Therapeutic Proteins and Peptides, 2018, pp. 1-59.

[11] H. J. Lee, A. McAuley, K. F. Schilke, J. McGuire, Molecular origins of surfactant-mediated stabilization of protein drugs, Adv Drug Deliv Rev, 63 (2011) 1160-1171.

[12] B. S. Chang, B. S. Kendrick, J. F. Carpenter, Surface-induced denaturation of proteins during freezing and its inhibition by surfactants, J Pharm Sci, 85 (1996) 1325-1330.

[13] N. B. Bam, J. L. Cleland, J. Yang, M. C. Manning, J. F. Carpenter, R. F. Kelley, T. W. Randolph ∥, Tween protects recombinant human growth hormone against agitation-induced damage via hydrophobic interactions, Journal of Pharmaceutical Sciences, 87 (1998) 1554-1559.

[14] P. Garidel, C. Hoffmann, A. Blume, A thermodynamic analysis of the binding interaction between polysorbate 20 and 80 with human serum albumins and immunoglobulins: a contribution to understand colloidal protein stabilisation, Biophys Chem, 143 (2009) 70-78.

[15] D. K. Chou, R. Krishnamurthy, T. W. Randolph, J. F. Carpenter, M. C. Manning, Effects of Tween 20 and Tween 80 on the stability of Albutropin during agitation, J Pharm Sci, 94 (2005) 1368-1381.

[16] S. Kiese, A. Papppenberger, W. Friess, H. C. Mahler, Shaken, not stirred: mechanical stress testing of an IgG1 antibody, J Pharm Sci, 97 (2008) 4347-4366.

[17] A. Gerhardt, A. C. McUmber, B. H. Nguyen, R. Lewus, D. K. Schwartz, J. F. Carpenter, T. W. Randolph, Surfactant Effects on Particle Generation in Antibody Formulations in Pre-filled Syringes, J Pharm Sci, 104 (2015) 4056-4064.

[18] K. A. Britt, D. K. Schwartz, C. Wurth, H. C. Mahler, J. F. Carpenter, T. W. Randolph, Excipient effects on humanized monoclonal antibody interactions with silicone oil emulsions, J Pharm Sci, 101 (2012) 4419-4432.

[19] J. Li, S. Pinnamaneni, Y. Quan, A. Jaiswal, F. I. Andersson, X. Zhang, Mechanistic Understanding of Protein-Silicone Oil Interactions, Pharmaceutical Research, 29 (2012) 1689-1697.

[20] J. F. Carpenter, B. S. Chang, W. Garzon-Rodriguez, T. W. Randolph, Rational Design of Stable Lyophilized Protein Formulations: Theory and Practice, in: J. F. Carpenter, M. C. Manning (Eds.) Rational Design of Stable Protein Formulations: Theory and Practice, Springer US, Boston, MA, 2002, pp. 109-133.

[21] W. Wang, S. Singh, D. L. Zeng, K. King, S. Nema, Antibody structure, instability, and formulation, J Pharm Sci, 96 (2007) 1-26.

[22] A. Hillgren, J. Lindgren, M. Aldén, Protection mechanism of Tween 80 during freeze-thawing of a model protein, LDH, Int. J. Pharm., 237 (2002) 57-69.

[23] W. R. Wasylaschuk, P. A. Harmon, G. Wagner, A. B. Harman, A. C. Templeton, H. Xu, R. A. Reed, Evaluation of hydroperoxides in common pharmaceutical excipients, J. Pharm. Sci., 96 (2007) 106-116.

[24] K. S. K. Ravuri, Polysorbate Degradation and Quality, in: N. W. Warne, H. -C. Mahler (Eds.) Challenges in Protein Product Development, Springer International Publishing, Cham, 2018, pp. 25-62.

[25] R. S. K. Kishore, S. Kiese, S. Fischer, A. Pappenberger, U. Grauschopf, H. -C. Mahler, The Degradation of Polysorbates 20 and 80 and its Potential Impact on the Stability of Biotherapeutics, Pharm. Res., 28 (2011) 1194-1210.

[26] R. S. K. Kishore, A. Pappenberger, I. B. Dauphin, A. Ross, B. Buergi, A. Staempfli, H. -C. Mahler, Degradation of Polysorbates 20 and 80: Studies on Thermal Autoxidation and Hydrolysis, J. Pharm. Sci., 100 (2011) 721-731.

[27] A. Tomlinson, B. Demeule, B. Lin, S. Yadav, Polysorbate 20 Degradation in Biopharmaceutical Formulations: Quantification of Free Fatty Acids, Characterization of Particulates, and Insights into the Degradation Mechanism, Mol Pharm, 12 (2015) 3805-3815.

[28] T. Hall, S. L. Sandefur, C. C. Frye, T. L. Tuley, L. Huang, Polysorbates 20 and 80 Degradation by Group XV Lysosomal Phospholipase A2 Isomer X1 in Monoclonal Antibody Formulations, J Pharm Sci, 105 (2016) 1633-1642.

[29] S. R. Labrenz, Ester hydrolysis of polysorbate 80 in mAb drug product: evidence in support of the hypothesized risk after the observation of visible particulate in mAb formulations, J Pharm Sci, 103 (2014) 2268-2277.

[30] N. Doshi, B. Demeule, S. Yadav, Understanding Particle Formation: Solubility of Free Fatty Acids as Polysorbate 20 Degradation Byproducts in Therapeutic Monoclonal Antibody Formulations, Mol Pharm, 12 (2015) 3792-3804.

[31] M. Saggu, J. Liu, A. Patel, Identification of Subvisible Particles in Biopharmaceutical Formulations Using Raman Spectroscopy Provides Insight into Polysorbate 20 Degradation Pathway, Pharm Res, 32 (2015) 2877-2888.

[32] T. Wang, A. Markham, S. J. Thomas, N. Wang, L. Huang, M. Clemens, N. Rajagopalan, Solution Stability of Poloxamer 188 Under Stress Conditions, J Pharm Sci, 108 (2019) 1264-1271.

[33] <2002_Erlandsson_Stability-indicating changes in poloxamers the degradation of.pdf>.

[34] L. S. Jones, A. Kaufmann, C. R. Middaugh, Silicone oil induced aggregation of proteins, J Pharm Sci, 94 (2005) 918-927.

[35] R. Thirumangalathu, S. Krishnan, M. S. Ricci, D. N. Brems, T. W. Randolph, J. F. Carpenter, Silicone oil- and agitation-induced aggregation of a monoclonal antibody in aqueous solution, J Pharm Sci, 98 (2009) 3167-3181.

[36] E. T. Maggio, Use of excipients to control aggregation in peptide and protein formulations, J. Excipients and Food Chem., 1 (2010) 9.

[37] R. A. Rifkin, E. T. Maggio, S. Dike, D. A. Kerr, M. Levy, n-Dodecyl-beta-D-maltoside inhibits aggregation of human interferon-beta-1b and reduces its immunogenicity, J Neuroimmune Pharmacol, 6 (2011) 158-162.

[38] E. T. Maggio, Polysorbates, peroxides, protein aggregation, and immunogenicity—a growing concern, J. Excipients and Food Chem., 1 (2012) 9.

[39] E. Söderlind, M. Wollbratt, C. von Corswant, The usefulness of sugar surfactants as solubilizing agents in parenteral formulations, International Journal of Pharmaceutics, 252 (2003) 61-71.

[40] L. Schiefelbein, M. Keller, F. Weissmann, M. Luber, F. Bracher, W. Friess, Synthesis, characterization and assessment of suitability of trehalose fatty acid esters as alternatives for polysorbates in protein formulation, Eur J Pharm Biopharm, 76 (2010) 342-350.

[41] J. S. Katz, Y. Tan, K. Kuppannan, Y. Song, D. J. Brennan, T. Young, L. Yao, S. Jordan, Amino-Acid-Incorporating Nonionic Surfactants for Stabilization of Protein Pharmaceuticals, ACS Biomaterials Science & Engineering, 2 (2016) 1093-1096.

[42] J. S. Katz, A. Nolin, B. A. Yezer, S. Jordan, Dynamic Properties of Novel Excipient Suggest Mechanism for Improved Performance in Liquid Stabilization of Protein Biologics, Mol Pharm, 16 (2019) 282-291.

[43] M. Agarkhed, C. O'Dell, M. C. Hsieh, J. Zhang, J. Goldstein, A. Srivastava, Effect of Surfactants on Mechanical, Thermal, and Photostability of a Monoclonal Antibody, AAPS PharmSciTech, 19 (2017) 79-92.

[44] M. Katakam, L. N. Bell, A. K. Banga, Effect of Surfactants on the Physical Stability of Recombinant Human Growth Hormone, Journal of Pharmaceutical Sciences, 84 (1995) 713-716.

[45] U.S. Food and Drug Administration. Inactive ingredient search for approved drug products, in, 2020.

[46] European Medicines Agengy, in, 2020.

[47] G. Thiagarajan, A. Semple, J. K. James, J. K. Cheung, M. Shameem, A comparison of biophysical characterization techniques in predicting monoclonal antibody stability, MAbs, 8 (2016) 1088-1097.

[48] D. B. Temel, P. Landsman, M. L. Brader, Orthogonal Methods for Characterizing the Unfolding of Therapeutic Monoclonal Antibodies: Differential Scanning Calorimetry, Isothermal Chemical Denaturation, and Intrinsic Fluorescence with Concomitant Static Light Scattering, Calorimetry, 567 (2016) 359-389.

[49] H. Svilenov, U. Markoja, G. Winter, Isothermal chemical denaturation as a complementary tool to overcome limitations of thermal differential scanning fluorimetry in predicting physical stability of protein formulations, European Journal of Pharmaceutics and Biopharmaceutics, 125 (2018) 106-113.

[50] W. Cheng, S. B. Joshi, F. He, D. N. Brems, B. He, B. A. Kerwin, D. B. Volkin, C. R. Middaugh, Comparison of high-throughput biophysical methods to identify stabilizing excipients for a model IgG2 monoclonal antibody: conformational stability and kinetic aggregation measurements, J Pharm Sci, 101 (2012) 1701-1720.

[51] A. C. McShan, P. Kei, J. A. Ji, D. C. Kim, Y. J. Wang, Hydrolysis of Polysorbate 20 and 80 by a Range of Carboxylester Hydrolases, PDA J Pharm Sci Technol, 70 (2016) 332-345.

[52] V. V. Bhaskar, A. Middha, P. Srivastava, S. Rajagopal, Liquid chromatography/tandem mass spectrometry method for quantitative estimation of solutol HS15 and its applications, J Pharm Anal, 5 (2015) 120-129.

[53] S. Fekete, K. Ganzler, J. Fekete, Simultaneous determination of polysorbate 20 and unbound polyethylene-glycol in protein solutions using new core-shell reversed phase column and condensation nucleation light scattering detection, J Chromatogr A, 1217 (2010) 6258-6266.

[54] M. Lei, J. Sugahara, D. Hewitt, D. Beane, R. Jayakar, C. Cornell, K. Skidmore, Y. H. Kao, J. Ji, The effects of membrane filters used in biopharmaceutical processes on the concentration and composition of polysorbate 20, Biotechnol Prog, 29 (2013) 1503-1511.

[55] K. T. Naidu, N. P. Prabhu, Protein-surfactant interaction: sodium dodecyl sulfate-induced unfolding of ribonuclease A, J Phys Chem B, 115 (2011) 14760-14767.

[56] J. H. Hansen, S. V. Petersen, K. K. Andersen, J. J. Enghild, T. Damhus, D. Otzen, Stable intermediates determine proteins' primary unfolding sites in the presence of surfactants, Biopolymers, 91(2009) 221-231.

[57] M. Jafari, F. Mehrnejad, F. Rahimi, S. M. Asghari, The Molecular Basis of the Sodium Dodecyl Sulfate Effect on Human Ubiquitin Structure: A Molecular Dynamics Simulation Study, Sci. Rep., 8 (2018) 2150.

[58] S. Kiese, A. Papppenberger, W. Friess, H. -C. Mahler, Shaken, Not Stirred: Mechanical Stress Testing of an IgG1 Antibody, J. Pharm. Sci., 97 (2008) 4347-4366.

[59] European Pharmacopeia 9.2, 2.2.1. Clarity and degree of opalescence of liquids. Strasbourg, France: The European Directorate for the Quality of Medicines & Health Care, (2017).

[60] European Pharmacopeia 9.0, 2.2.2. Degree of coloration of liquids. Strasbourg, France: The European Directorate for the Quality of Medicines & Health Care, (2008).

[61] European Pharmacopeia 9.0, 2.9.19. Particulate contamination: sub-visible particles. Strasbourg, France: The European Directorate for the Quality of Medicines & Health Care, (2011).

[62] USP <787>, Subvisible particulate matter in therapeutic protein injections, Pharmacopeia Forum, 38 (2012).

[63] S. Amrhein, S. Suhm, J. Hubbuch, Surface tension determination by means of liquid handling stations, Engineering in Life Sciences, 16 (2016) 532-537.

[64] K. Holmberg, Natural Surfactants, Current Opinion in Colloid & Interface Science, 6 (2001) 148-159.

[65] B. M. Folmer, M. Svensson, K. Holmberg, W. Brown, The Physicochemical Behavior of Phytosterol Ethoxylates, J. Colloid Interface Sci., 213 (1999) 112-120.

[66] C. Grapentin, C. Müller, R. S. K. Kishore, M. Adler, I. ElBialy, W. Friess, J. Huwyler, T. A. Khan, Protein-Polydimethylsiloxane Particles in Liquid Vial Monoclonal Antibody Formulations Containing Poloxamer 188, J. Pharm. Sci., (2020).

1. A liquid pharmaceutical composition comprising a protein and one or more surfactant(s) selected from the group consisting of TPGS, PVA, T1107, Px338, Px407, TMN-6, 15-S-15, Chol-PEG, and SL.

2. The composition according to claim 1, wherein the protein is an antibody, an immunoconjugate, or an antibody fragment.

3. The composition according to claim 1, further comprising one or more of a pharmaceutically acceptable excipient or carrier.

4. The composition according to claim 1, wherein the surfactant(s) is/are present in a concentration of ≤1 mg/mL; or in a concentration range from 0.001 mg/mL to 0.01 mg/mL; or from 0.01 mg/mL to 0.1 mg/mL; or from 0.1 mg/mL to 1.0 mg/mL.

5. The use of one or more surfactants selected from TPGS, PVA, T1107, Px338, Px407, TMN-6, 15-S-15, Chol-PEG, and SL in the manufacture of a liquid pharmaceutical composition further comprising a protein.

6. The use of one or more surfactants selected from TPGS, PVA, T1107, Px338, Px407, TMN-6, 15-S-15, Chol-PEG, and SL for stabilizing a protein and preventing the formation of visible particles in a liquid pharmaceutical composition comprising said protein, upon storage.

7. The composition according to claim 2, wherein the surfactant(s) is/are present in a concentration of ≤1 mg/mL; or in a concentration range from 0.001 mg/mL to 0.01 mg/mL; or from 0.01 mg/mL to 0.1 mg/mL; or from 0.1 mg/mL to 1.0 mg/mL.

8. The composition according to claim 3, wherein the surfactant(s) is/are present in a concentration of ≤1 mg/mL; or in a concentration range from 0.001 mg/mL to 0.01 mg/mL; or from 0.01 mg/mL to 0.1 mg/mL; or from 0.1 mg/mL to 1.0 mg/mL.
